(19) United States
(12) Patent Application Publication
(10) Pub. No.: US 2013/0164850 A1
(43) Pub. Date: Jun. 27, 2013

(54) METHOD FOR TARGETED GENOMIC EVENTS IN ALGAE

(71) Applicant: David Sourdive, Levallois-Perret (FR)

(72) Inventor: David Sourdive, Levallois-Perret (FR)

(21) Appl. No.: 13/813,705

(22) PCT Filed: Aug. 1, 2011
(86) PCT No.: PCT/IB2011/002605

§ 371 Date: Mar. 7, 2013

Related U.S. Application Data
(60) Provisional application No. 61/370,017, filed on Aug. 2, 2010.

(51) Int. Cl. C12N 9/16

(52) U.S. Cl. CPC C12N 9/16
USPC 435/441; 435/257.2

(57) ABSTRACT

The invention relates to endonucleases cleaving DNA target sequences from algae genomes, to appropriate vectors encoding such endonucleases, to cells or to algae modified by such vectors and to the use of these endonucleases and products derived therefrom for targeted genomic engineering in algae.

### CROSS-REFERENCE TO RELATED APPLICATIONS

The present PCT International patent application claims priority to U.S. provisional patent application 61/370,017, filed on Aug. 2, 2010, the contents of which are hereby incorporated by reference in their entirety.

### BACKGROUND OF THE INVENTION

1. Field of the Invention

The invention relates to endonucleases cleaving DNA target sequences from algae genomes, to appropriate vectors encoding such endonucleases, to cells or to algae modified by such vectors and to the use of these endonucleases and products derived therefrom for targeted genomic engineering in algae.

2. Discussion of the Background

Although algae have been used as a food source by humans for centuries, the significance of their biotechnological interest, especially of microalgae, appeared only in recent decades. Applications of algal products range from simple biomass production for food, feed and fuels to valuable products such as cosmetics, pharmaceuticals, pigments, sugar polymers and food supplements. For most of these applications, the market is still developing and considering the enormous biodiversity of microalgae and development of genetic engineering, this group of organisms represents one of the most promising sources of new products and applications.

Algae can be found in nearly all aquatic and terrestrial ecosystems, most of them being poorly investigated at the biochemical level, while showing a huge biodiversity and various morphologies ranging from picoplankton species to large kelp (Norton, T. A., Melkonian, M., & Andersen, R. A. (1996) Phycologia 35, 308-326).

Several algal species such as Dunaliella bardawil, Haematococcus pluvialis and Chlorella vulgaris have already been exploited extensively in the past for biotechnological purposes, especially as feed, as a source of pigments like β-carotene or astaxanthin or as food supplements (Steinbrenner, J. & Sandmann, G. (2006) Appl. Environ. Microbiol. 72, 7477-7484; Mogedas, B., Casal, C., Forjan, E., & Vilchez, C. (2009) J Biosci Bioeng 108, 47-51.).

Most of these organisms are green algae that belonging to a group more related to land plants than other algal groups (Palmer, J. D., Soltis, D. E., & Chase, M. W. (2004) Am. J. Bot. 91, 1437-1445).

Chromophytic algae on the other hand only recently moved into the forefront and their biochemistry and genetics have been studied just in the recent years. They comprise important groups like the brown algae, diatoms, xanthophytes, eustigmatophytes and others, but also the colourless oomycetes (Tyler, B. M., Tripathy, S., Zhang, X., Dehal, P., Jiang, R. H. Y., Aerts, A., Arredondo, F. D., Baxter, L., Bensasson, D., Beynon, J. L. et al. (2006) Science 313, 1261-1266.). Research on chromophytic algae received a strong boost after publication of several genomes including those of the diatoms Thalassiosira pseudonana (Armbrust, E. V., Berges, J. A., Bowler, C., Green, B. R., Martinez, D., Putnam, N. H., Zhou, S., Allen, A. E., Apt, K. E., Bechner, M. et al. (2004) Science 306, 79-86.) and Phaeodactylum tricornutum (Bowler, C., Allen, A. E., Badger, J. H., Grimwood, J., Jabbari, K., Kuo, A., Maheswari, U., Martens, C., Maumus, F., Otillar, R. P. et al. (2008) Nature. 456, 239-244.).

Genomes of other algae such as Fragilariopsis cylindrus, the haptophyte Emiliania huxleyi (http://genome.jgi-psf.org/Emihul/Emihul.home.html) and the brown alga Ectocarpus siliculosus (in preparation, http://www.cns.fr/spip/-Ectocarpus-siliculosus-.html) are presently studied.

With a large set of genes that originate from bacteria by lateral gene transfer (Bowler, C., Allen, A. E., Badger, J. H., Grimwood, J., Jabbari, K., Kuo, A., Maheswari, U., Martens, C., Maumus, F., Otillar, R. P. et al. (2008) Nature. 456, 239-244.), chromophytic algae demonstrate an enormous genetic complexity and metabolic potential.

Nannochloropsis, for example, is a genus of algae comprising approximately six species inside the eustigmatophytes group. Nannochloropsis is able to build up a high concentration of a range of pigments such as astaxanthin, zeaxanthin and canthaxanthin. These algae have a very simple ultrastructure that is reduced compared to neighbouring taxa. It is considered as a promising alga for industrial applications because of its ability to accumulate high levels of polyunsaturated fatty acids. It is also mainly used as an energy-rich food source for fish larvae and rotifers.

Diatoms, as another example, are one of the most ecologically successful unicellular phytoplankton on the planet, being responsible for approximately 20% of global carbon fixation, representing a major participant in the marine food web. There are two major potential commercial or technological applications of diatoms. First, Diatoms are able to accumulate abundant amounts of lipid suitable for conversion to liquid fuels and because of their high potential to produce large quantities of lipids and good growth efficiencies, they are considered as one of the best classes of algae for renewable biofuel production. Second, Diatoms have a cell wall consisting of silica (silica exoskeletons called frustules) with intricated and ornate structures on the nano- to micro-scale. These structures exceed the diversity and the complexity capable by man-made synthetic approaches, and Diatoms are being developed as a source of materials mainly for nanotechnological applications (Lusic et al Advanced Functional Materials 2006).

Although some genetic tools to explore microalgal technology are available such as several sequences of genomes and the ability to be genetically transformed, few genome engineering tools exist which considerably limits the use of these organisms for various biotechnological applications. With the current Diatom transformation technology, transformed DNA randomly integrates into the genome, which results in different expression levels for different transformants using the same DNA construct; identifying the highest level of expression can require time-consuming and tedious screening methods.

Any commercial or technological application involving algae will be greatly facilitated by having the ability to perform targeted genomic manipulations ranging from targeted insertions in chosen loci (knock-in), considered or not as “safe harbors” for gene addition (i.e. a loci allowing safe expression of a transgene), to targeted gene knock-out, allele swap, substitutions, marker excisions and deletions, within algae genomes. Moreover, it would be extremely advantageous if these tools allowed targeted genomic manipulations with a high efficacy.

Meganucleases, also referred to as homing endonucleases, were the first endonucleases used to induce double-strand breaks and recombination in living cells (Rouet et al. PNAS 1994 91:6064-6068; Rouet et al. Mol Cell Biol. 1994 14 :8096-8106; Choulika et al. Mol Cell Biol. 1995 15 :1968-1973; Puchta et al. PNAS 1996 93 :5055-5060). However, their use has long been limited by their narrow specificity. Although several hundred natural meganucleases had been identified over the past years, this diversity was still largely insufficient to address genome complexity, and the probability of finding a meganuclease cleavage site within a gene of interest is still extremely low. These findings highlighted the need for artificial endonucleases with tailored specificities, cleaving chosen sequences with the same selectivity as wild-type endonucleases.

Meganucleases have emerged as the scaffolds of choice for creating genome engineering tools cutting a desired target sequence (Paques et al. Curr Gen Ther. 2007 7:49-66). Combinatorial assembly processes allowing to engineer meganucleases with modified specificities has been described by Arnould et al. J Mol Biol. 2006 355:443-458; Arnould et al. J Mol Biol. 2007 371:49-65; Smith et al. NAR 2006 34:e149; Grizot et al. NAR 2009 37:5405). Briefly, these processes rely on the identification of locally engineered variants with a substrate specificity that differs from the substrate specificity of the wild-type meganuclease by only a few nucleotides. Up to four sets of mutations identified in such proteins can then be assembled in new proteins in order to generate new meganucleases with entirely redesigned binding interfaces.

These processes require two steps, wherein different sets of mutations are first assembled into homodimeric variants cleaving palindromic targets. Two homodimers can then be co-expressed in order to generate heterodimeric meganucleases cleaving the chosen non palindromic target. The first step of this process remains the most challenging one, and one cannot know in advance whether a meganuclease cleaving a given locus could be obtained with absolute certainty. Indeed, not all sequences are equally likely to be cleaved by engineered meganucleases, and in certain cases, meganuclease engineering can prove difficult (Galetto et al. Expert Opin Biol Ther. 2009 9:1289-303).

The inventors have now found new endonucleases cleaving targets within algal genomes that could be used as tools allowing efficient targeted DNA modifications of these genomes, thereby considerably facilitating the handling of these organisms for various biotechnological applications.

### SUMMARY OF THE INVENTION

Therefore, the present invention concerns endonuclease cleaving targets within algal genomes that could be used as tools allowing efficient targeted genomic engineering of these genomes, thereby considerably facilitating the use of these organisms for various biotechnological applications.

Thus, methods are provided to obtain cultivated algae with engineered genomes at specific targeted sites by using endonuclease variants, thereby considerably increasing usability of these organisms for various biotechnological applications. These provided methods range from targeted insertions in chosen loci (knock-in), considered or not as “safe harbors” for gene addition (i.e. a loci allowing safe expression of a transgene), to targeted gene knock-out, allele swap, substitutions, marker excisions, deletions, inside algae genomes as non-limiting examples of genome engineering.

The above topics highlight certain aspects of the invention. Additional objects, aspects and embodiments of the invention are found in the following detailed description of the invention.

### BRIEF DESCRIPTION OF THE FIGURES

In addition to the preceding features, the invention further comprises other features which will emerge from the description which follows, which refers to examples illustrating endonuclease variants and their uses according to the invention, as well as to the appended drawings. A more complete appreciation of the invention and many of the expected advantages thereof will be readily obtained as the same becomes better understood by reference to the following Figures in conjunction with the detailed description below.

FIG. 1: Illustration of different meganuclease-induced types of recombination events leading to stable and precise genomic modifications. A. Targeted integration (insertion of a transgene) through gene conversion; this approach can also be used for gene knock out by homologous recombination. B. Knock-out of a gene through Non-Homologous-End-Joining (NHEJ) initiated by a unique double-stranded break. C. Knock-out of a gene through NHEJ initiated by two double-stranded breaks (gene excision).

FIG. 2: Modular structure of homing endonucleases and the combinatorial approach for custom meganucleases design A. Tridimensional structure of the I-CreI homing endonuclease bound to its DNA target. The catalytic core is surrounded by two αββαββα folds, forming a saddle-shaped interaction interface above the DNA major groove. B. A combinatorial process for meganuclease engineering: Four separable DNA binding subdomains (boxed) could be identified in the I-CreI scaffold, an homodimeric meganuclease, that binds and cleaves a palindromic target. Each subdomain can be engineered specifically (boxed), resulting in novel meganucleases cleaving locally altered palindromic targets. Two different subdomains can be combined within a “half meganuclease”, a homodimeric meganuclease binding a palindromic target. Two such “half meganucleases” can be co-expressed to form a heterodimeric custom meganuclease that will cleave a novel non palindromic target. Additional steps of engineering (by random or targeted mutagenesis and screening) are often required at this stage to optimize the activity of meganucleases, resulting in a refined meganuclease. In the final version, the two refined monomers can be connected by a linker to make a single-chain meganuclease, as described in Grizot et al. (2009).

FIG. 3: Schematic of the STA6 locus from Chlamydomonas reinhardtii. The coding sequences and mRNA sequences are shown, with intervening introns in white. The target positions for 16 meganucleases able to specifically recognize and cleave this locus are also depicted.

FIG. 4: Sequences and locations of targeted sites in the STA6 gene from Chlamydomonas reinhardtii (Gene bank accession number: NW001843572).

FIG. 5: Sequences and locations of meganucleases targeted sites in Phaeodactylum tricornutum genome (genomic sequences for analysis were found at: http://genome.jgi-psf.org/Phatr2/Phatr2.home.html).

FIG. 6: Sequences and locations of meganucleases targeted sites in Thalassiosira pseudonana genome (genomic sequences for analysis were found at: http://genome.jgi-psf. org/Thaps3/Thaps3.home.html).

FIG. 7: Sequences and locations of meganucleases targeted sites in Chlorella (NC64A) genome (genomic sequences for analysis were found at: http://genome.jgi-psf.org/Ch1NC64A_{—}1/Ch1NC64A_{—}1.home.html).

FIG. 8: Theoretic map of the pThpse-LHCF9p-TP7-LHCF9-3′ expression plasmid (SEQ ID NO: 71). LHCF9p=diatom specific promoter region. SC-TP7=single chain TP7 meganuclease ORF. LHCF9-3′ poly(A) signal. I-CreI and I-SceI=target sequences of I-CreI and I-SceI respectively. The rest of the plasmid is a pUC19.

FIG. 9: Theoretic map of the pThpse-LHCF9p-NAT-LHCF9-3′ expression plasmid (SEQ ID NO: 72). LHCF9p=diatom specific promoter region. nat1=nat1 gene ORF (nourseopthricin acetyl transferase). LHCF9-3′ poly(A) signal. I-CreI and I-SceI=target sequences of I-CreI and I-SceI respectively. The rest of the plasmid is a pUC19.

FIG. 10: A) TP7-nat selection plate showing positive colonies; B) Agarose gel electrophoresis of 44 PCR colony screenings from TP7 electroporation. White arrowheads indicate colonies used for deep-sequencing assay.

FIG. 11: Agarose gel electrophoresis of 13 cDNA amplifications (RT-PCR) showing the presence of full length mRNAs of the MN TP7 in some of the strains. Light and contrast if the picture were modified to detect the paler bands.

FIG. 12: Western blot of 13 strains (the strain names are reported below the tracks). As positive control 250 ng of I-CreI purified monomeric protein was used. The band recorded at 40 Kd represents the dimerization of I-CreI. As negative control, T. pseudonana wild type protein extract was used.

FIG. 13: Theoretic map of the TP7-KI matrix (SEQ ID NO: 73). LHCF9p=diatom specific promoter region. SC-TP7=single chain TP7 meganuclease ORF (SEQ ID NO: 69). LHCF9-3′ poly(A) signal. I-CreI and I-SceI=target sequences of I-CreI and I-SceI respectively. The rest of the plasmid is a pUC19.

### DETAILED DESCRIPTION OF THE INVENTION

Unless specifically defined herein below, all technical and scientific terms used herein have the same meaning as commonly understood by a skilled artisan in the fields of gene therapy, biochemistry, genetics and molecular biology. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will overrule. Further, the materials, methods, and examples are illustrative only and are not intended to be limiting, unless otherwise specified.

The present invention concerns endonucleases cleaving targets within algal genomes that could be used as tools allowing efficient targeted genomic engineering of these genomes, thereby considerably facilitating the handling of these organisms for various biotechnological applications.

As used herein, the term “endonuclease” refers to any wild-type or variant enzyme capable of catalyzing the hydrolysis (cleavage) of bonds between nucleic acids within a DNA or RNA molecule, preferably a DNA molecule. The endonucleases according to the present invention do not cleave the DNA or RNA molecule irrespective of its sequence, but recognize and cleave the DNA or RNA molecule at specific polynucleotide sequences, further referred to as “target sequences”, “target sites”, “recognition sites” or “recognition sequences”. Target sequences recognized and cleaved by an endonuclease according to the invention are referred to as target sequences according to the invention.

The endonuclease according to the invention can for example be a homing endonuclease (Paques et al. Curr Gen Ther. 2007 7:49-66), a chimeric Zinc-Finger nuclease (ZFN) resulting from the fusion of engineered zinc-finger domains with the catalytic domain of a restriction enzyme such as Fokl (Porteus et al. Nat Biotechnol. 2005 23:967-973) or a chemical endonuclease (Arimondo et al. Mol Cell Biol. 2006 26:324-333; Simon et al. NAR 2008 36:3531-3538; Eisenschmidt et al. NAR 2005 33 :7039-7047; Cannata et al. PNAS 2008 105:9576-9581). For chemical endonucleases, a chemical or peptidic cleaver is conjugated either to a polymer of nucleic acids or to another DNA recognizing a specific target sequence, thereby targeting the cleavage activity to a specific sequence.

The endonuclease according to the invention is preferably a homing endonuclease, also known as meganuclease (s). Such homing endonucleases are well-known to the art (see e.g. Stoddard, Quarterly Reviews of Biophysics, 2006, 38:49-95). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Examples of such endonuclease include I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I, I-MsoI.

In a preferred embodiment, the homing endonuclease according to the invention is a LAGLIDADG endonuclease such as I-SceI, I-CreI, I-CeuI, I-MsoI, and I-DmoI.

In a most preferred embodiment, said LAGLIDADG endonuclease is I-CreI. Wild-type I-CreI is a homodimeric homing endonuclease that is capable of cleaving a 22 to 24 bp double-stranded target sequence. The sequence of a wild-type monomer of I-CreI includes the sequence shown as SEQ ID NO: 1 (which corresponds to the I-CreI sequence of pdb accession number 1g9y).

In the present patent application, the I-CreI variants may comprise an additional alanine after the first methionine of the wild type I-CreI sequence, and three additional amino acid residues at the C-terminal extremity (see sequence of SEQ ID NO: 2). These three additional amino acid residues consist of two additional alanine residues and one aspartic acid residue after the final proline of the wild type I-CreI sequence. These additional residues do not affect the properties of the enzyme. For the sake of clarity, these additional residues do not affect the numbering of the residues in I-CreI or variants thereof. More specifically, the numbering used herein exclusively refers to the position of residues in the wild type I-CreI enzyme of SEQ ID NO: 1. For instance, the second residue of wild-type I-CreI is in fact the third residue of a variant of SEQ ID NO: 2 since this variant comprises an additional alanine after the first methionine.

In the present application, I-CreI variants may be homodimers (meganuclease comprising two identical monomers) or heterodimers (meganuclease comprising two non-identical monomers). It is understood that the scope of the present invention also encompasses the I-CreI variants per se, including heterodimers (WO2006097854), obligate heterodimers (WO2008093249) and single chain meganucleases (WO03078619 and WO2009095793) as non limiting examples, able to cleave one of the sequence targets in the algal genome. The invention also encompasses hybrid variant per se composed of two monomers from different origins (WO03078619).

The invention encompasses both wild-type and variant endonucleases. In a preferred embodiment, the endonuclease according to the invention is a “variant” endonuclease, i.e. an endonuclease that does not naturally exist in nature and that is obtained by genetic engineering or by random mutagenesis. The variant endonuclease according to the invention can for example be obtained by substitution of at least one residue in the amino acid sequence of a wild-type, endonuclease with a different amino acid. Said substitution(s) can for example be introduced by site-directed mutagenesis and/or by random mutagenesis. In the frame of the present invention, such variant endonucleases remain functional, i.e. they retain the capacity of recognizing and specifically cleaving a target sequence.

The variant endonuclease according to the invention cleaves a target sequence that is different from the target sequence of the corresponding wild-type endonuclease. For example, the target sequence of a variant I-CreI endonuclease is different from the sequence of SEQ ID NO:3 (palindromic sequence C1221 derived from the wild-type IcreI recognition site). Methods for obtaining such variant endonucleases with novel specificities are well-known in the art.

The present invention is based on the finding that such variant endonucleases with novel specificities can be used to allow efficient targeted genomic engineering within the algal genomes, thereby considerably increasing the usability of these organisms for various biotechnological applications.

In the frame of the present invention, “algae” or “algae cells” or “cells”, refer to different species of algae that can be used as hosts for genomic transformation using the meganucleases of the present invention, polynucleotides and vectors encoding them, including for example without limitation one or more algae selected from Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

By “gene” it is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns and a 3′ untranslated region. The gene may further be comprised of terminators, enhancers and/or silencers.

By “genome” it is meant the entire genetic material contained in a cell such as nuclear genome, chloroplastic genome, mitochondrial genome . . . .

By “nearest genes” it is meant the two genes that are located closest to the target sequence, centromeric and telomeric to the target sequence respectively.

As used herein, the term “locus” is the specific physical location of a DNA sequence (e.g. of a gene) on a chromosome. As used in this specification, the term “locus” usually refers to the specific physical location of an endonuclease's target sequence on a chromosome. Such a locus, which comprises a target sequence that is recognized and cleaved by an endonuclease according to the invention, is referred to as “locus according to the invention”.

By “site of interest” it is meant a locus inside a genome containing an endonuclease target sequence or a putative endonuclease target sequence for an engineered endonuclease with a modified specificity such as said endonuclease is able to cleave said target inside said site of interest to achieve a targeted genomic event.

As used herein, the term “transgene” refers to a sequence inserted at a site of interest in an algal genome. Preferably, it refers to a sequence encoding a polypeptide. Preferably, the polypeptide encoded by the transgene is either not expressed, or expressed but not biologically active, in the algae or algal cells in which the transgene is inserted. Most preferably, the transgene encodes a polypeptide useful for increasing the usability and the commercial value of algae. Also, the transgene can be a sequence inserted at a site of interest in an algae genome for producing an interfering RNA.

As used herein, the expressions “gene of interest,” “nucleotide sequence of interest”, “nucleic acid of interest” or “sequence of interest” refer to any nucleotide or nucleic acid sequence that encodes a protein or other molecule that is desirable for expression in an algal cell (e.g. for production of the protein or other biological molecule [e.g., an RNA product like interfering RNA as a non limiting example] in the target cell). The nucleotide sequence of interest is generally operatively linked to other sequences which are needed for its expression, e.g., a promoter. Further, the sequence itself may be regulatory in nature and thus of interest for expression in the target cell.

By “homologous” it is meant a sequence with enough identity to another one to lead to homologous recombination between sequences, more particularly having at least 95% identity, preferably 97% identity and more preferably 99%.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.

By “mutation” it is meant the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

In a preferred embodiment, the present invention provides endonuclease variants to perform targeted gene knock-out in algae. Gene knock-out is the most powerful tool for determining gene function or permanently modifying the phenotypic characteristics of a cell. The repair of double strand DNA breaks (DSB) in mammalian cells occurs via the distinct mechanisms of homology directed repair (HDR) or NHEJ. Although HDR typically uses the sister chromatid of the damaged DNA as a template from which to perform perfect repair of the genetic lesion, NHEJ is an imperfect repair process that often results in changes to the DNA sequence at the site of the DSB. During NHEJ, the cleaved DNA is further resected by exonuclease activity, and more bases may be added in an imprecise manner before the two ends of the damaged DNA are rejoined. The subject-matter of the present invention is also a method for making a targeted knock-out in algae wherein a targeted double-stranded cleavage at a site of interest inside the algae genome is induced by an endonuclease, and repaired by the non-homologous end-joining pathway (NHEJ), resulting in loss of gene function. Preferably, the endonuclease of the present invention is a meganuclease.

In a particular aspect of this embodiment the knocked-out algae is made by introducing into said algae, an endonuclease as defined above, so as to induce a double stranded cleavage at a site of interest of the genome comprising a DNA recognition and cleavage site of said endonuclease, and thereby generate genetically modified algae knocked-out for the gene located at this site of interest of the genome, said modified algae having repaired the double-strands break by NHEJ, and isolating said genetically modified algae knocked-out by any appropriate means.

In another particular aspect of this embodiment the knocked-out algae is made by introducing into an algae, 1) an endonuclease as defined above, so as to induce a double stranded cleavage at a site of interest of the genome comprising a DNA recognition and cleavage site of said endonuclease, 2) a knock-out template to be introduced is flanked by sequences sharing homologies with the region surrounding the genomic DNA cleavage site and thereby generates genetically modified algae knocked-out for the gene located at this site of interest of the genome, said modified algae having repaired the double-strands break by HDR, and isolating said genetically modified algae knocked-out by any appropriate means.

In another preferred embodiment, the present invention provides endonuclease variants to target sequence insertions (knock-in) into chosen loci of the genome. In this embodiment the knocked-in algae is made by introducing into an algae, 1) an endonuclease as defined above, so as to induce a double stranded cleavage at a site of interest for sequence insertion in the genome comprising a DNA recognition and cleavage site of said endonuclease, 2) a knock-in template to be introduced flanked by sequences sharing homologies with the region surrounding the genomic DNA cleavage site and thereby generating genetically modified algae at this site of interest of the genome, said modified algae having repaired the double-strands break by HDR, and isolating said genetically modified algae by any appropriate means.

By “sequence insertion”, it is intended the introduction into a target genome of an exogenous nucleotidic sequence.

By “targeting DNA construct/minimal repair matrix/repair matrix/template (knock-out template or knock-in template)” it is intended a DNA construct comprising a first and second portion of sequences which are homologous to regions 5′ and 3′ of the DNA target in situ, at a site of interest in the algal genome. The DNA construct also comprises a third portion positioned between the first and second portion which can comprise some homology with the corresponding DNA sequence in situ (in the cases of allele/promoter swap as non-limiting examples) or alternatively can comprise no homology with the regions 5′ and 3′ of the DNA target in situ (insertion of a selectable marker). Following cleavage of the DNA target, a homologous recombination event is stimulated between the genome containing the targeted gene or part of the targeted gene and the repair matrix, wherein the genomic sequence containing the DNA target is replaced by the third portion of the repair matrix and a variable part of the first and second portions of the repair matrix. The repair matrix can also be endogenous such as a chromosomal sequence of interest. The chromosomal sequence of interest can be either located on the same chromosome as the genomic locus of interest, or on a different chromosome.

Preferably, homologous sequences of at least 50 bp, preferably more than 100 bp and more preferably more than 200 bp are used. Therefore, the targeting DNA construct is preferably from 200 bp to 6000 bp, more preferably from 1000 bp to 2000 bp. Indeed, shared DNA homologies are located in regions flanking upstream and downstream the site of the break and the DNA sequence to be introduced should be located between the two arms. The targeting construct comprises advantageously a positive selection marker between the two homology arms and eventually a negative selection marker upstream of the first homology arm or downstream of the second homology arm. The marker(s) allow(s) the selection of algae having inserted the sequence of interest by homologous recombination at the target site.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

In a particular aspect of this embodiment, sequence insertions can be used to modify a targeted existing gene, by correction or replacement of said gene (allele swap as a non-limiting example), or to up or down regulate the expression of the targeted gene (promoter swap as non-limiting example), said targeted gene correction or replacement conferring one or several commercially desirable traits.

In another particular aspect of this embodiment, sequence insertions can be used to introduce new sequences or genes of interest increasing the potential exploitation of algae by conferring them commercially desirable traits for various biotechnological applications.

As non-limiting examples, traits that can be engineered in Algae and that are comprised in the scope of the present invention can be traits related to: —Quorum Sensing (QS) (QS allows cell-to-cell communication: sensing the environment. This system well-described in bacteria, uses a chemical signaling mechanism to coordinate expression of various genes when a sufficient population of bacteria has been reached. Several QS signaling molecules have been identified in bacteria, most notably the N-acylhomoserine lactone (AHL) family, and the Autoinducer 1 (acylated homoserine lactone) and Autoinducer 2 (a furanosyl borate diester) compounds (Teplitski et al., Plant Physiology, 2004). Interestingly, certain algae such as the red macroalgae Delisea pulchra, secrete GS mimic molecules (furanone compounds), that interfere with the bacterial AHL QS sensing signals, and thus can be used to prevent growth of harmful bacterial pathogens (Hentzer and Givskov, The Journal of Clinical Investigation, 2003; Williams, Microbiology, 2007). AHL mimic substances that activate QS have been studied in the model algal organism Chlamydomonas reinhardtii, which is amenable to genetic and molecular studies (the sequence is also available: http://www.biology.duke.edu/chlamy_genome/). The identification and targeting of these algal mimic compounds can be applied to the prevention of marine bacterial/algal biofilm development and algal blooms, and in the case of the algal furanones from marine algae, to create antipathogenic drugs (Kuehl et al., 2009, Antimicrobial Agents and Chemotherapy). Finally, certain toxic algae (such as Gymnodinium catenatum) living with marine bacteria (that release neurotoxins infecting shellfish and causing paralytic shellfish poisoning), require the QS signals from bacteria (sideopherones and borate) to know there is enough iron available, —Secretion of hydrocarbons (without limitation, lipids, isoprenoids, polyunsaturated aldehydes as source of alkanes or alkenes, production of polymers such as alginates), —Fatty acid composition (lipid branching), —Lipid accumulation (biofuel production). A target for increasing biofuel production has been isolated in the green algae Chlamydomonas, (Li et al; 2010, Metabolic Engineering). In this study, inhibition of starch synthesis in a starchless mutant within the gene encoding for ADP-glucose pyrophosphorylase led to hyper-accumulation of fatty acids and triacylglycerol (TAG). —Lipids and antibacterial, therauptic applications: The polyunsaturated fatty acid from P. tricornutum, called eicosapentaenoic acid (EPA), was also recently shown to act as an effective anti-bacterial reagent against pathogenic bacteria such as the multi-drug resistant Staphyloccus aureus (MRSA), not susceptible to most known antibiotics (Desbois et al; 2009, Mar Biotechnol), —Photosynthesis (additional pigments to enlarge useful light wavelengths), —Pigment production (Carotenoids and Phycobiliproteins as non-limiting examples), —Herbicide resistance, —Mercury volatilization, —Frustule composition and organization (nanostructured materials and devices). Diatoms display a diverse array of silicon structures at the nano- to millimeter scale and diatom nanotechnology can be applied to the fields of biophotonics, photoluminescence, microfluids, silica sequestering, multiscale porosity, silica sequestering of proteins, detection of trace gases, computer design and controlled drug delivery (Gordon et al; 2008, Trends in Biotechnology). The silica deposition vesicles (SDV), silicon transport vesicles, clathrin pits, and microtubules and silaffins (long chain polyamines) are major components of the silicon transport pathway, necessary for silica precipitation. —Biosafety issues, such as, in a non-limiting example, to avoid a transgene of interest to disseminate in natural ecosystems.

Some genetic elements that are related to said previous engineerable traits can be, without limitation: Acylhomoserine Lactone (AHL) (Jun Sum Kim et al Biotechnol and Bioprocess Engineering 2007), delta(12)-fatty acid dehydrogenase (fad2), fatty acid desaturase thioesterase (TE), Tla1 antennae. Similar acting genes, or antenna mutant (e.g. chlorophyll a oxygenase-CAO),(advantageous in high light only), Aldolase and TPI D-fructose 1,6-bisphosphatase/sedoheptulose 1,7-bisphosphatase, Blue Fluorescent protein, Overexpressed cystathione γ-synthase, Elevated dihydropicolinate synthase and suppressed lysine ketobutyrate reductase/saccharopine dehydrogenase. 5-enolpyruvylshikimate-3-phosphate synthase (EPSPS), Phytoene desaturase, glyphosate oxidoreductase, acetolactate synthase, Nitrilase, phosphinothricin N-acetyltransferase, 4-hydroxyphenyl-pyruvate-dioxygenase (HPPD), Protoporphyrinogen oxidase (PPO or protox), Glutamine synthetase, Helicase, replicase, viral coat protein, exo-alpha-sialidase (involved in N-glycan degradation and sphingolipid metabolism), silaffins and silicic acid transporters, furanones, PtCPF1 protein from P. tricornutum (and all members of the cryptochrome/photolyase family (CPF), Diatom Si transporters (SIT) (Thamatrakoln and Hildebrand, 2008, Plant Physiology), Proteins involved in manipulation of silicon (Mock et al; 2008, PNAS), FCP proteins (fucoxanthin chlorophyll a/c proteins), RuBisCo, Silaffins, Silaffin transporters, ADP-glucose pyrophosphorylase and EPA (eicosapentaenoic acid).

Other uses of this particular aspect of the invention, include the insertion of sequences that can be an interfering sequence i.e a sequence silencing genes of interest or respective products of said genes, per se (RNA interference process well-known in the art) or by the interfering agent coded by said interfering sequence, these interfering sequences conferring one or several commercially desirable traits by their silencing actions. These interfering sequences can be one or more sequences selected from siRNAs, shRNAs, miRNAs, cDNAs.

As mentioned above the term “interfering sequence” refers to a sequence able to silence a gene per se or by the “interfering agent” encoded by this interfering sequence. As a non-limiting example, said interfering sequence can code for a protein inhibitor i.e the interfering agent in this case, this protein inhibitor being able to interact with and inhibit a targeted enzyme, this silencing process conferring to the algae host a commercially advantageous trait.

Gene silencing by RNAi has been characterized and used as a tool to generate targeted gene knockdown or knockout mutants in Phaeodactylum tricornutum (De Riso, V. et al 2009). However, as previously used this RNAi approach cannot be used to create strains containing stable gene knock-down or knock-outs. Use of meganucleases according to the present invention is ideally suited for this.

Interfering RNAs (iRNAs) include, miRNAs, siRNAs and shRNAs; an interfering RNA is also an interfering agent as described above.

As shown at least in mammalian cells, the enzyme Dicer cleaves long dsRNAs into short-interfering RNAs (siRNAs) of approximately 21-23 nucleotides. One of the two siRNA strands is then incorporated into an RNA-induced silencing complex (RISC). RISC compares these “guide RNAs” to RNAs in the cell and efficiently cleaves target RNAs containing sequences that are perfectly, or nearly perfectly complementary to the guide RNA. “iRNA construct” also includes nucleic acid preparation designed to achieve a RNA interference effect, such as expression vectors able of giving rise to transcripts which form dsRNAs or hairpin RNA in cells, and or transcripts which can produce siRNAs in vivo.

A “short interfering RNA” or “siRNA” comprises a RNA duplex (double-stranded region) and can further comprises one or two single-stranded overhangs, 3′ or 5′ overhangs. Each molecule of the duplex can comprise between 17 and 29 nucleotides, including 17, 18, 19, 20, 21, 22, 23, 24, 25, 26, 27, 28, and 29 nucleotides. siRNAs can additionally be chemically modified.

“MicroRNAs” or “miRNAs” are endogenously encoded RNAs that are about 22-nucleotide-long, that post-transcriptionally regulate target genes and are generally expressed in a highly tissue-specific or developmental-stage-specific fashion. At least more than 200 distinct miRNAs have been identified in plants and animals. These small regulatory RNAs are believed to serve important biological functions by two predominant modes of action: (1) by repressing the translation of target mRNAs, and (2) through RNA interference, that means cleavage and degradation of mRNAs. In this latter case, miRNAs function analogously to siRNAs. miRNAs are first transcribed as part of a long, largely single-stranded primary transcript (pri-miRNA) [Lee et al., 2002, EMBO J. 21: 4663-4670]. This pri-miRNA transcript is generally and possibly invariably, synthesized by RNA polymerase II and therefore is polyadenylated and may be spliced. It contains an approximate 80-nucleotides long hairpin structure that encodes for the mature, approximately 22-nucleotides miRNA part of one arm of the stem. In animal cells, this primary transcript is cleaved by a nuclear RNaseIII-type enzyme called Drosha (Lee et al, 2003, Nature 425:415-419) to liberate a hairpin mRNA precursor, or pre-miRNA of about-65 nucleotides long. This pre-miRNA is then exported to the cytoplasm by exportin-5 and the GTP-bound form of the Ran cofactor (Yi et al, 2003, Genes and Development 17:3011-3016). Once in the cytoplasm, the pre-miRNA is further processed by Dicer, another RNaseIII enzyme to produce a duplex of about-22 nucleotides base pairs long that is structurally identical to a siRNA duplex (Hutvagner et al, 2001, Science 293:834-838). The binding of protein components of the RISC, or RISC cofactors, to the duplex results in incorporation of the mature, single-stranded miRNA into a RISC or RISC-like protein complex, while the other strand of the duplex is degraded (Bartel et al, 2004, Cell 116: 281-297). Thus, one can design and express artificial miRNAs based on the features of existing miRNA genes. The miR-30 (microRNA 30) architecture can be used to express miRNAs (or siRNAs) from RNA polymerase II promoter-based expression plasmids (Zeng et al, Methods enzymol. 392:371-380). In some instances the precursor miRNA molecules may include more than one stem-loop structure. The multiple stem-loop structures may be linked to one another through a linker, such as, for example, a nucleic acid linker, a miRNA flanking sequence, other molecules, or some combination thereof.

A “short hairpin RNA (shRNA)” refers to a segment of RNA that is complementary to a portion of a target gene (complementary to one or more transcripts of a target gene), and has a stem-loop (hairpin) structure, and which can be used to silence gene expression. A “stem-loop structure” refers to a nucleic acid having a secondary structure that includes a region of nucleotides which are known or predicted to form a double strand (stem portion) that is linked on one side by a region of predominantly single-stranded nucleotides (loop portion). The term “hairpin” is also used herein to refer to stem-loop structures.

In another particular aspect of this embodiment, the present invention provides endonuclease variants to insert genes at targeted chosen loci in algal genomes that can be considered as “safe harbors” for gene addition i.e. a loci allowing safe and stable expression of a transgene. The present invention is based on the finding that such variant endonucleases with novel specificities can be used for inserting a gene into a “safe harbor” locus of the genome of a algae. In a preferred embodiment, the locus according to the invention further allows stable expression of the transgene. In another preferred embodiment, the target sequence according to the invention is only present once within the genome of said algae. Ideally, insertion into a good safe harbor locus should have no impact on the expression of other genes. In a preferred aspect of this embodiment, a locus for targeted insertion is chosen close to a gene that is essential for survival of the targeted algae, said transgene inserted at this locus becoming genetically linked to this essential gene and the probability of their independent segregation from each other becoming extremely low. In this preferred aspect of this embodiment, said gene essential for the survival of the targeted algae is a housekeeping gene. In a non-limiting list, housekeeping genes that are comprised in the scope of the present invention are those required for the maintenance of basal cellular function, like as non-limiting examples, transcription factors, translation factors (tRNA synthetases, RNA binding proteins), ribosomal proteins, RNA polymerases, processing proteins, Heat Shock Proteins, histones, cell cycle genes, metabolism genes (carbohydrate metabolism, citric acid cycle, lipid of fatty acid metabolism, amino acid metabolism, nitrogen metabolism, urea cycle, polyamine biosynthesis pathway, nucleotide synthesis), structural genes (cytoskeleton, organelle synthesis), genes from chloroplast and from photosynthesis, carbon fixation, cell wall synthesis pathway and clathrin-mediated endocytosis.

Testing these properties is a multi-step process, and a first pre-screening of candidate safe harbor loci by bioinformatic means is desirable. One can thus first identify loci in which targeted insertion is unlikely to result in insertional mutagenesis.

In addition, in another specific embodiment, insertion of a genetic element into said locus does not substantially modify the phenotype of said algae (except for the phenotype due to expression of the genetic element). By “phenotype” it is meant an algae's or a algae cell's observable traits. The phenotype includes viability, growth, resistance or sensitivity to various marker genes, environmental and chemical signals, etc. . . . .

Once such a safe harbor locus according to the invention has been selected, one can then (i) either construct a variant endonuclease specifically recognizing and cleaving a target sequence located within said locus, or (ii) determine whether a known wild-type endonuclease is capable of cleaving a target sequence located within said locus. Alternatively, once a safe harbor locus according to the invention has been selected, the skilled in the art can insert therein a target sequence that is recognized and cleaved by a known wild-type or variant endonuclease.

Therefore, the invention is drawn to a method for obtaining an endonuclease suitable for safely inserting a transgene into the genome of an algae for example without substantially modifying (i) expression of the nearest genes, and/or (ii) the cellular proliferation and/or the growth rate of the cell, tissue or individual.

In another preferred embodiment, the present invention provides endonuclease variants to induce single-stranded annealing (SSA). When tandemly repeated homologous sequences surround a DSB, an efficient mode of DSB repair can be intra-chromosomal recombination by SSA between the two directly repeated homologous sequences, leading to the physical elimination of one repeat and all the sequences between the repeats. SSA is a powerful approach to excise sequences from the chromosome.

Site-specific recombinases such as Cre-lox and Flp recombinase systems have also been widely used in many cell types. Although these systems are efficient to perform marker removal, for example, their big drawback is that the final recombination event contains the exogenous or foreign recombination target site (typically a loxP site) which is not desirable in terms of Genetically Modified Organisms (GMO) issues, and remains functional, impeding future re-use of the same system in the cell. In addition, this exogenous footprint can lead to genomic instabilities and further chromosomal rearrangements.

Endonuclease-induced SSA-based excision, in contrast, efficiently leads to removal of, for example, marker sequences, leading to stable and precise recombination correction events, without leaving behind exogenous sequences. In other words, if one introduces a marker, with short regions of flanking homology, these sequences can then be later removed, leaving only the native wild type sequence, without any “scar” on the genome. This occurs by the highly efficient SSA recombination pathway. Marker removal by endonuclease induced SSA provides a major advantage in terms of generating non-GM strains or species. In addition, the marker can be repeatedly re-used again, which is an important issue in diatoms, since they lack a variety of different selection markers.

Only few publications refer to selection markers usable in Diatoms. Dunahay et al 1995 and Zaslayskaia et al 2001 report the use of the neomycin phosphotransferase II (nptII), that inactivates G418 by phosphorylation, in Cyclotella cryptic, Navicula saprophila and Phaeodactylum tricornutum species. Falciatore et al 1999, Fischer et al 1999 and Zaslayskaia et al 2001 report the use of the Zeocin resistance gene (Sh ble), acting by stoichiometric binding, in Phaeodactylum tricornutum and Cylindrotheca fusiformis species. In Zaslayskaia et al 2001, the use of N-acetyltransferase 1 gene (Nat1) conferring the resistance to Nourseothricin by enzymatic acetylation is reported in Phaeodactylum tricornutum and Thalassiosira pseudonana. It is understood that use of the previous specific selectable markers are comprised in the scope of the present invention and that use of other genes encoding other selectable markers including, for example and without limitation, genes that participate in antibiotic resistance are also comprised in the scope of the present invention.

Marker removal by the use of meganuclease provides a major advantage in terms of generating non-GM strains or species or by the fact that the few positive selection markers available in algae can be repeatedly used.

In another preferred embodiment, endonuclease variants provided in the present invention allow “transgene stacking”, i.e the insertion of multiple transgenes into the same, chosen, locus in the genome of an algae. Such targeted locations are referred to as “landing pads” in safe places within the genome. Endonuclease variants in the present invention allow flexible consecutive and reproducible sequence insertion into the same locus of any species of algae. In a particular aspect of this preferred embodiment, endonuclease variants in the present invention allow the link of multiple traits/genes in a recipient genome, i.e different sequences, alleles or traits, identified in separate algae strains or isolates, can be precisely re-introduced within a single industrial strain, without the need for sexual crossing.

In another preferred embodiment, endonuclease variants provided in the present invention allow “pathway engineering”. Since endonucleases in the present invention allow a broad range of genomic modifications (allele swap, gene stacking, promoter swap, gene knock-in or knock-out, inducible sequence pop-out as non-limiting examples), metabolic pathway engineering, increasing the usability and the commercial value of algae, is comprised in the scope of the present invention.

In another preferred embodiment, endonuclease variants provided in the present invention target sequences selected from the group consisting of the SEQ ID NO 4 to 19 from the genome of Chlamydomonas reinhardtii, SEQ ID NO 20 to 39 from the genome of Phaeodactylum tricornutum, SEQ ID NO 40 to 58 from the genome of Thalassiosira pseudonana and from the group consisting of the SEQ ID NO 59 to 68 from the genome of Chlorella (NC64A).

The subject-matter of the present invention is also a polynucleotide fragment encoding a variant of an endonuclease as defined above. Preferably, the subject-matter of the present invention is also a polynucleotide fragment encoding a variant meganuclease as defined above; said polynucleotide may encode for instance one monomer of a homodimeric or heterodimeric variant, or two domains/monomers of a single-chain meganuclease or any variants as defined above. It is understood that the subject-matter of the present invention is also a polynucleotide fragment encoding one of the variant species as defined above, obtained by any well-known method in the art.

The subject-matter of the present invention is also a recombinant vector for the expression of an endonuclease variant as defined above. The subject-matter of the present invention is also a recombinant vector for the expression of any variant according to the invention. The recombinant vector comprises at least one polynucleotide fragment encoding any as defined above.

By “vector” is intended to mean a nucleic acid molecule capable of transporting another nucleic acid to which it has been linked. A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those skilled in the art and commercially available. Some useful vectors include, for example without limitation, pGEM13z. pGEMT and pGEMTEasy {Promega, Madison, Wis.); pSTBluel (EMD Chemicals Inc. San Diego, Calif.); and pcDNA3.1, pCR4-TOPO, pCR-TOPO-II, pCRBlunt-II-TOPO (Invitrogen, Carlsbad, Calif.).

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli. Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is a heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-O-D-thiogalacto-pyranoside (IPTG) and eukaryotic heat shock promoter which is induced by increased temperature.

In some embodiments, the vector for the expression of the endonucleases according to the invention can be operably linked to an algal-specific promoter. In some embodiments, the algal-specific promoter is an inducible promoter. In some embodiments, the algal-specific promoter is a constitutive promoter. Promoters that can be used include, for example without limitation, a Pptcal promoter (the CO2 responsive promoter of the chloroplastic carbonic anyhydrase gene, ptcal, from P. tricornutum), a NIT1 promoter, an AMT1 promoter, an AMT2 promoter, an AMT4 promoter, a RH1 promoter, a cauliflower mosaic virus 35S promoter, a tobacco mosaic virus promoter, a simian virus 40 promoter, a ubiquitin promoter, a PBCV-I VP54 promoter, or functional fragments thereof, or any other suitable promoter sequence known to those skilled in the art.

In another most preferred embodiment according to the present invention the vector is a shuttle vector, which can both propagate in E. coli (the construct containing an appropriate selectable marker and origin of replication) and be compatible for propagation or integration in the genome of the selected algae.

According to another advantageous embodiment of said vector, it includes a targeting construct comprising sequences sharing homologies with the region surrounding the targeted genomic DNA cleavage site in algae as defined above.

For instance, said sequence sharing homologies with the regions surrounding the genomic DNA cleavage site of the variant is a fragment of the targeted genomic DNA. Alternatively, the vector encoding for an endonuclease variant/single-chain meganuclease and the vector comprising the targeting construct are different vectors.

Endonucleases provided in the present invention can be delivered in various formats: DNA, messenger RNA, or even as a protein.

A variety of different methods are known for the introduction of DNA into host cell nuclei or chloroplasts. In various embodiments, the vectors can be introduced into algae nuclei by, for example without limitation, electroporation, particle inflow gun bombardment, or magnetophoresis. The latter is a nucleic acid introduction technology using the processes of magnetophoresis and nanotechnology fabrication of micro-sized linear magnets (Kuehnle et al., U.S. Pat. No. 6,706,394; 2004; Kuehnle et al., U.S. Pat. No. 5,516,670; 1996) that proved amenable to effective chloroplast engineering in freshwater Chlamydomonas, improving plastid transformation efficiency by two orders of magnitude over the state-of the-art of biolistics (Champagne et al., Magnetophoresis for pathway engineering in green cells. Metabolic engineering V: Genome to Product, Engineering Conferences International Lake Tahoe Calif., Abstracts pp 76; 2004). Polyethylene glycol treatment of protoplasts is another technique that can be used to transform cells (Maliga, P. Plastid Transformation in Higher Plants. Annu. Rev. Plant Biol. 55:294; 2004). In various embodiments, the transformation methods can be coupled with one or more methods for visualization or quantification of nucleic acid introduction to one or more algae.

Direct microinjection of purified endonucleases of the present invention in algae can be considered. Also appropriate mixtures commercially available for protein transfection can be used to introduce endonucleases in algae according to the present invention. More broadly, any means known in the art to allow delivery inside cells or subcellular compartments of agents/chemicals and molecules (proteins) can be used to introduce endonucleases in algae according to the present invention including liposomal delivery means, polymeric carriers, chemical carriers, lipoplexes, polyplexes, dendrimers, nanoparticles, emulsion, natural endocytosis or phagocytose pathway as non-limiting examples.

The subject matter of the present invention is also a kit for making knock-out/knock-in in algae comprising at least an endonuclease and/or one expression vector, as defined above. Preferably, the subject matter of the present invention is also a kit for making knock-out/knock-in in algae comprising at least a meganuclease and/or one expression vector, as defined above. More preferably, the kit further comprises a targeting DNA comprising a sequence that inactivates the targeted gene flanked by sequences sharing homologies with the region of the targeted gene surrounding the DNA cleavage site of said meganuclease. In addition, for making knocked-in algae, the kit includes also a vector comprising a sequence of interest to be introduced in the genome of said algae and eventually a selectable marker gene, as defined above.

In accordance with some embodiments of the present invention, and combinations between these embodiments, bioprocess algae containing commercially desirable traits by the use of one or more endonuclease variants according to the present invention, are comprised in the scope of the present invention. Particularly, is comprised in the scope of the present invention a targeted genome engineered algae (i.e an algae whose genome has been modified at a targeted site of interest) wherein said algae genome contains at least one gene modified by one or more endonuclease variants according to the present invention. More particularly, is comprised in the scope of the present invention a targeted genome engineered algae encoding at least one gene conferring an advantageous trait for biotechnological applications, selected from the group of genes encoding quorum sensing, secretion of hydrocarbons, fatty acid composition, lipids accumulation, enhanced photosynthesis, pigments production, mercury volatilization, frustule composition or organization, mitigation genes in a non-exhaustive list, wherein said at least one gene has been introduced by one or more endonuclease variants according to the present invention.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of”, “chosen from” and the like include mixtures of the specified materials.

Where a numerical limit or range is stated herein, the endpoints are included. Also, all values and sub-ranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Microalgae are considered as one of the best alternatives to produce advanced liquid fuels such as biodiesel and bio-jet fuels, due to their capacity to synthesize large quantities of triacylglycerols (TAG). The ‘microalgae for fuel’ concept has been explored over the last 12 years, but previous attempts to increase lipid production by focusing strictly on fatty acid biosynthesis (Dunahay et al. 1996; Marillia et al. 2003; Roesler et al. 1997), increasing the availability of glycerol (Vigeolas et al; 2007) or over-expression of genes in the TAG biosynthesis pathway (Bouvier-Nave et al. 2000; Jako et al. 2001; Weselake et al. 2008; Zheng et al; 2008; Zou et al. 1997) have all shown limited success.

In green microalgae, starch synthesis shares common carbon precursors with lipid synthesis. STA6 codes for the catalytic small sub-unit of ADP-glucose pyrophosphorylase (AGPase), which is a well-conserved protein within all algal and photosynthetic organisms and is necessary for ADP-glucose synthesis (Zabawinski et al., J. of Bacteriology, 2001). Furthermore, mutants within this locus fail to accumulate starch.

Recent evidence in the model microalga organism, Chlamydomonas reinhardtii, shows that shunting of photosynthetic carbon precursors from starch to TAG synthesis, by inactivation of STA6, results in a 10-fold increase of fatty acid and TAG lipids (Li et al; Metabolic Engineering, 2010; Li et al; Biotechnology and Bioengineering, 2010). Therefore, STA6 is an important target for understanding how carbon partioning, through the inactivation of starch, may increase lipid production, ideally without compromising viability and growth rates.

Therefore, a first main aspect of this invention concerns the engineering of meganucleases targeting the STA6 gene to create a stable loss of gene function. Another aspect of this invention uses meganucleases for targeted knock-in of constructs containing site-specific substitutions within STA6 to create precise disruptions (i.e. a 1 or 2 bp nucleotide substitution), which should define new separation of function alleles, that is mutations that specifically increase lipid production significantly without compromising viability, growth or yield. It can also be useful to add marker constructs either within or just in cis to the STA6 locus, so that the strain containing the mutated locus can be easily followed and maintained upon sexual crosses or during other molecular and genetic manipulations. A second main aspect of this invention illustrated in Example 2 addresses how meganucleases can target entire genomes, including intergenic regions to be used as landing pads for targeted insertions of interesting target constructs.

### Example 1

### Engineering Meganucleases Targeting the STA6 Gene in Chlamydomonas reinhardtii

A) Disruption (Knock-Out) of the STA6 Gene Using Meganucleases Targeting the STA6 Gene without a Repair Matrix.

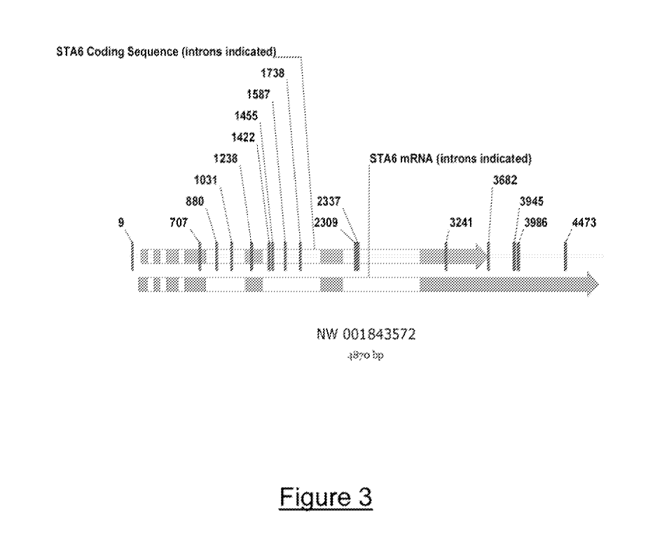

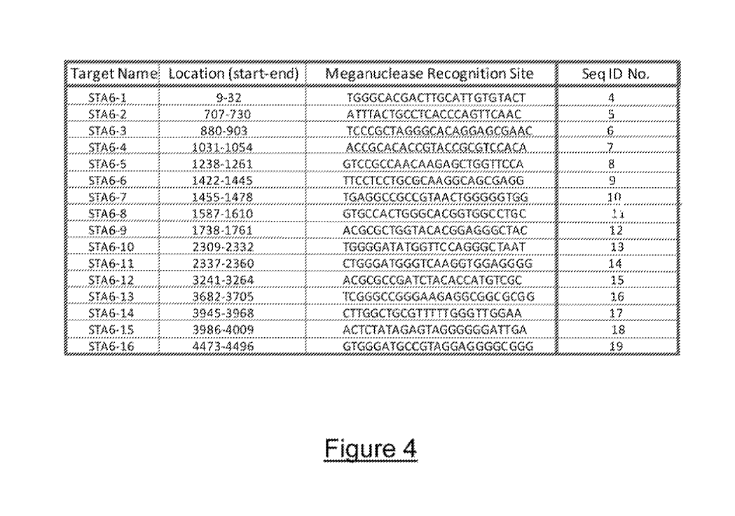

One strategy envisioned to knock-out the STA6 gene, involves mutating the coding sequence by non homologous end joining (NHEJ), using a STA6 meganuclease targeting a sequence within the open reading frame (see FIGS. 3 and 4 and Table 1 below). In this case, the STA6 meganucleases recognizing exons 4 (STA6-2), 5 (STA6-5) or 7 (STA6-12) can be used to create a double-strand break in these exonic sites. In the absence of homology or a repair matrix, the double-stranded ends will either rejoin perfectly, or imperfectly, using micro-homologies near the break site. Imperfect end-joining, gives rise to small deletions or insertions within the open reading frame, and therefore generates loss of gene function. Lipid content in sta6 mutant strains compared to wild-type strains can be measured by testing the ability to produce TAG under both favorable (low light and nitrogen replete) and stress conditions that induce TAG synthesis (high-light and nitrogen-starved) (Hu et al., 2008, Plant J.). Using degenerate primers, homologous to this sequence, one can easily identify, clone, and sequence STA6 homologs in other algal species and identify meganucleases to perform the same type of knock-out approach.

TABLE 1
Sequences and locations of targeted sites in the
STA6 gene from Chlamydomonas reinhardtii
(Gene bank accession number: NW001843572).

| | Location | | |
|---|---|---|---|
| Target Name | (start-end) | Meganuclease Recognition Site | Seq ID No. |
| STA6-1 | 9-32 | TGGGCACGACTTGCATTGTGTACT | 4 |
| STA6-2 | 707-730 | ATTTACTGCCTCACCCAGTTCAAC | 5 |
| STA6-3 | 880-903 | TCCCGCTAGGGCACAGGAGCGAAC | 6 |
| STA6-4 | 1031-1054 | ACCGCACACCGTACCGCGTCCACA | 7 |
| STA6-5 | 1238-1261 | GTCCGCCAACAAGAGCTGGTTCCA | 8 |
| STA6-6 | 1422-1445 | TTCCTCCTGCGCAAGGCAGCGAGG | 9 |
| STA6-7 | 1455-1478 | TGAGGCCGCCGTAACTGGGGGTGG | 10 |
| STA6-8 | 1587-1610 | GTGCCACTGGGCACGGTGGCCTGC | 11 |
| STA6-9 | 1738-1761 | ACGCGCTGGTACACGGAGGGCTAC | 12 |
| STA6-10 | 2309-2332 | TGGGGATATGGTTCCAGGGCTAAT | 13 |
| STA6-11 | 2337-2360 | CTGGGATGGGTCAAGGTGGAGGGG | 14 |
| STA6-12 | 3241-3264 | ACGCGCCGATCTACACCATGTCGC | 15 |
| STA6-13 | 3682-3705 | TCGGGCCGGGAAGAGGCGGCGCGG | 16 |
| STA6-14 | 3945-3968 | CTTGGCTGCGTTTTTGGGTTGGAA | 17 |
| STA6-15 | 3986-4009 | ACTCTATAGAGTAGGGGGGATTGA | 18 |
| STA6-16 | 4473-4496 | GTGGGATGCCGTAGGAGGGGCGGG | 19 |

B) Complete Disruption (Knock-Out) and Gene Replacements (Knock-in) to Create New Alleles within the STA6 Gene or Promoter (Using Meganucleases and a KO/Substitution Repair Matrix).

A second strategy to generate a loss of gene function, takes advantage of a knock-out repair matrix. This consists of disrupting a large region or even the entire STA6 open reading frame, using the two STA6 meganucleases STA6-1 and STA6-13 that respectively target just upstream and downstream of the open-reading frame and a knock-out repair matrix to generate a large deletion or disruption. To create a complete deletion with no insertion, the repair matrix is designed using sequences of flanking homology (typically 500-1000 bp are used) outside and/or just within the STA6 open reading frame and deleted for the coding exon regions. One can also design the repair knock-out construct, with for example, a marker resistance gene such as the phytoene desaturase gene, (Frommolt et al. 2008, Mol. Biol. Evol.; Junchao et al., 2008, J. of Phycology), embedded between the same flanking homologous sequences for targeted gene replacement.

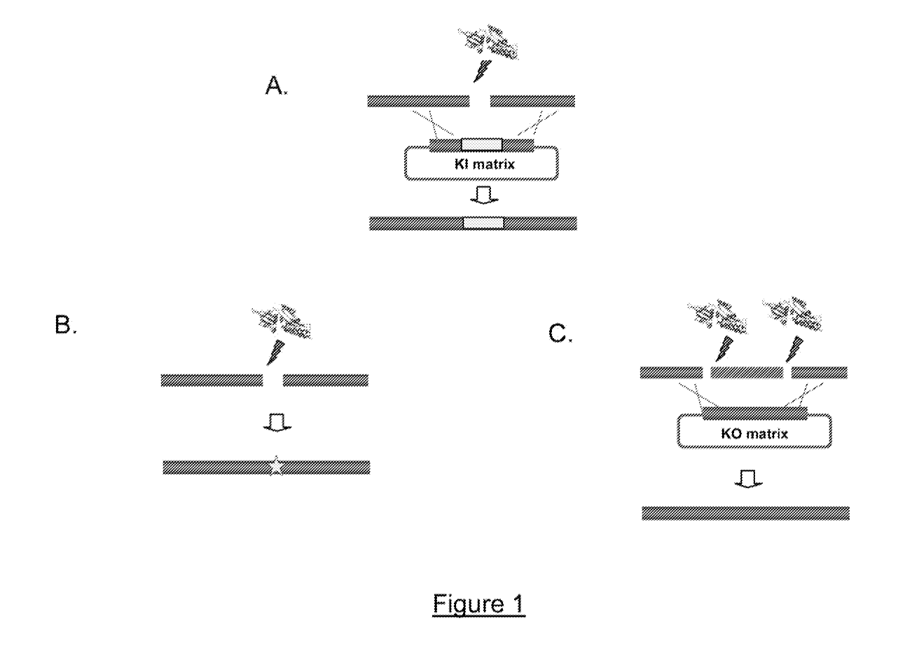

More subtle gene targeted replacements can be designed, using one or several of the meganucleases listed in FIG. 1 and repair matrixes that contain specific DNA substitutions, or even randomly mutagenized sequences flanked by perfect homology to the STA6 locus. Site-specific substitutions within the STA6 locus may result in mutants with very different phenotypes, in terms of lipid production, growth and viability, as compared to a full deletion (null) construct. One can also modify gene expression, using the meganuclease targeting the promoter region (STA6-1), and a repair matrix containing a strong constitutive promoter, an attenuated version of the same endogenous promoter, or an inducible promoter, such as the heat-shock-inducible promoter hsp70, from the green alga as characterized in Chlamydomonas and Volvox (Cheng, et al., 2006, Gene, pp. 112-120).

### Example 2

### Engineering Meganucleases to Target Different Sites in the Genome of the Diatom Phaeodactylum tricornutum for Targeted Integration (Use of Intergenic Regions as Safe Landing Pads for Insertions Constructs)

Example 1 provided an illustration of how to genetically modify and create various types of disruptions and substitutions within one defined locus, using meganucleases. With the recent advent of entire genome sequencing projects, meganucleases can be engineered to target sequences not only locally but globally. In this respect, target sites within intergenic regions, can be used as safe harbors or landing pads for insertion of interesting target sequences and constructs. In another words, these intergenic regions can be considered as safe because they should not perturb other neighbouring chromosomal genes either by disruption or by modifying their expression.

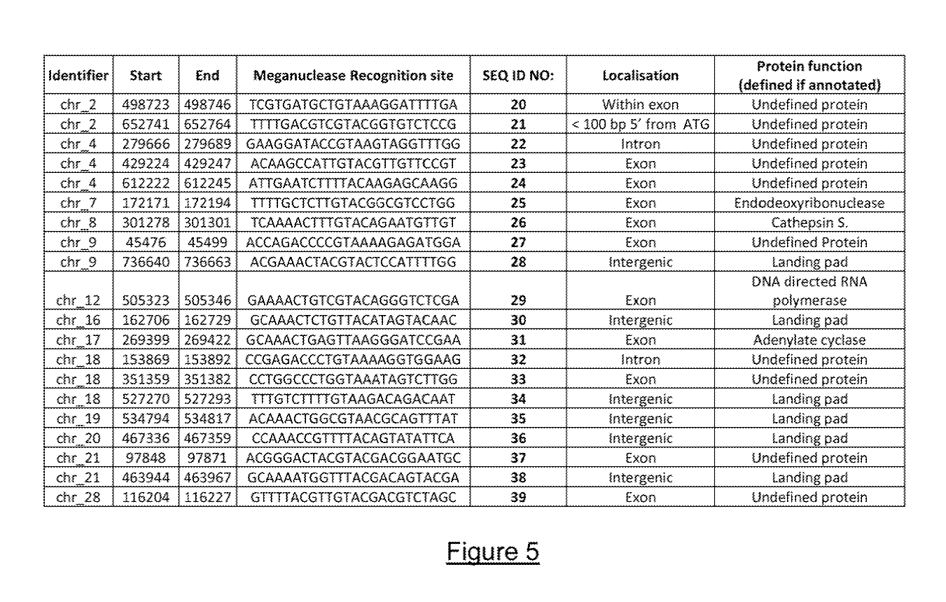

In FIG. 5, different meganucleases cleaving sites within genes or intergenic regions located in the P. tricornutum genome have been identified. The six intergenic sites identified can be used as sites for insertion of marker constructs, RNA silencing constructs, or other sequences of interest.

Recently the fatty acid eicosapentaenoic acid (EPA) from Phaeodactylum tricornutum was shown to display antibacterial activity in vitro against the pathogenic multidrug-resistant Staphyloccus aureus (MRSA), not susceptible to most antibiotics (Desbois, et al., 2009, Mar Biotechnol, pp. 45-52). In addition, EPA from P. tricornutum is able to inhibit the growth of the fish and shellfish pathogen Listonella anguillarum, arguing that overexpression of EPA could be useful in controlling disease in the mariculture industry and for human health purposes, when conventional antibiotics are not suitable. Studies involving EPA in this Diatom have only been performed at a biochemical level using purified extracts of this molecule. For the moment, many putative genomic EPA targets exist in P. tricornutum, but thus far none have been cloned, isolated and targeted for genomic modification. As EPA is difficult to extract and purify, it would be interesting to create EPA cDNA constructs either from algal species, plants or mammals, and insert such constructs using meganucleases at intergenic, safe site, as indicated in FIG. 5. In this way, algae can serve as a tool for understanding EPA function, and perhaps to overexpress this molecule for downstream therapeutic uses.

### Example 3

### Engineering Meganucleases to Target Different Sites in the Genome of the Diatom Thalassiosira pseudonana for Targeted Integration (Use of Intergenic Regions as Safe Landing Pads for Insertions Constructs).

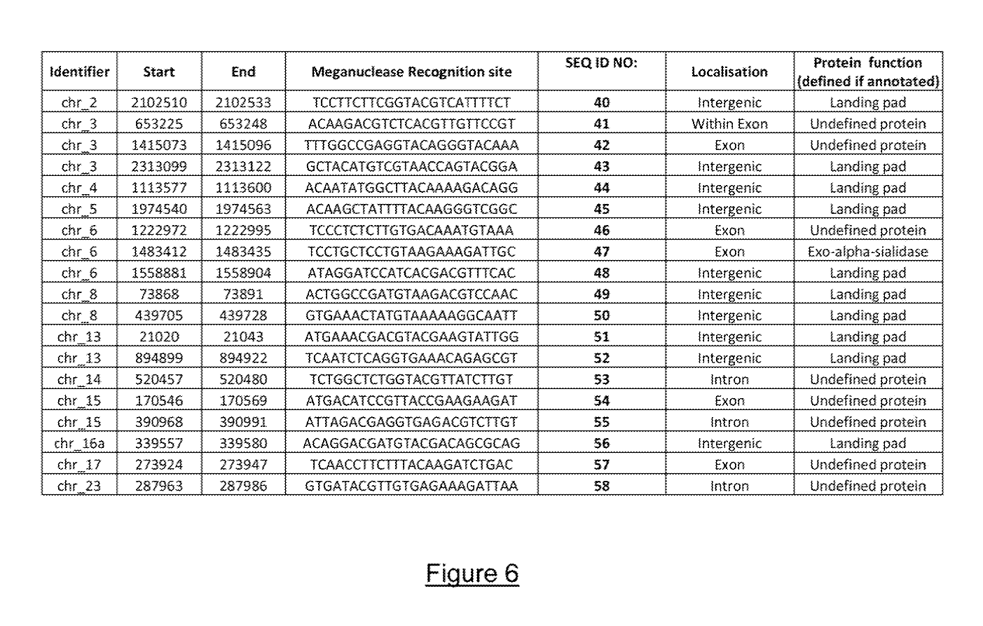

In FIG. 6, different meganucleases cleaving sites within genes or intergenic regions located in the Thalassiosira pseudonana genome have been identified. The ten intergenic sites identified can be used as sites for insertion of marker constructs, RNA silencing constructs, or other sequences of interest.

### Example 4

### Engineering Meganucleases to Target Different Sites in the Genome of the Diatom Chlorella (NC64A) for Targeted Integration (Use of Intergenic Regions as Safe Landing Pads for Insertions Constructs)

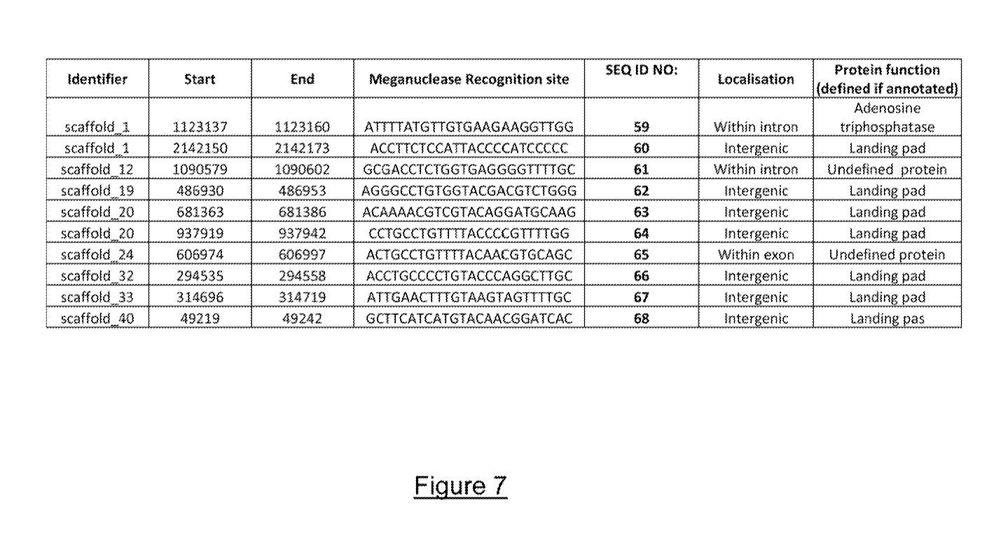

In FIG. 7, different meganucleases cleaving sites within genes or intergenic regions located in the Chlorella (NC64A) genome have been identified. The seven intergenic sites identified can be used as sites for insertion of marker constructs, RNA silencing constructs, or other sequences of interest.

### Example 5

### Targeted Genomic Events in Thalassiosira pseudonana Using TP7 Engineered Meganucleases

1) Subcloning of TP7 Meganuclease and Nourseothricin Acetyl Transferase Gene (nat1) and Open Reading Frames into Diatom Specific Expression Plasmids

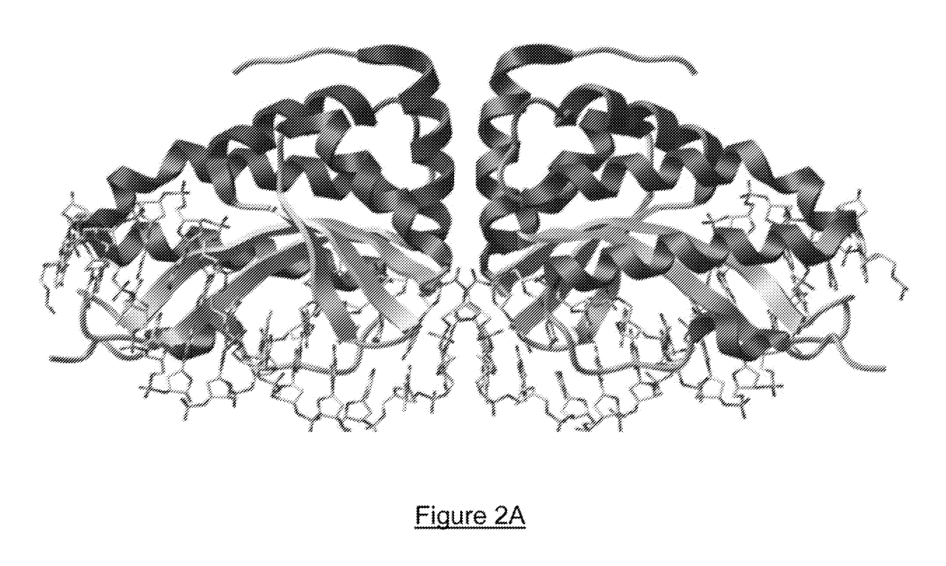

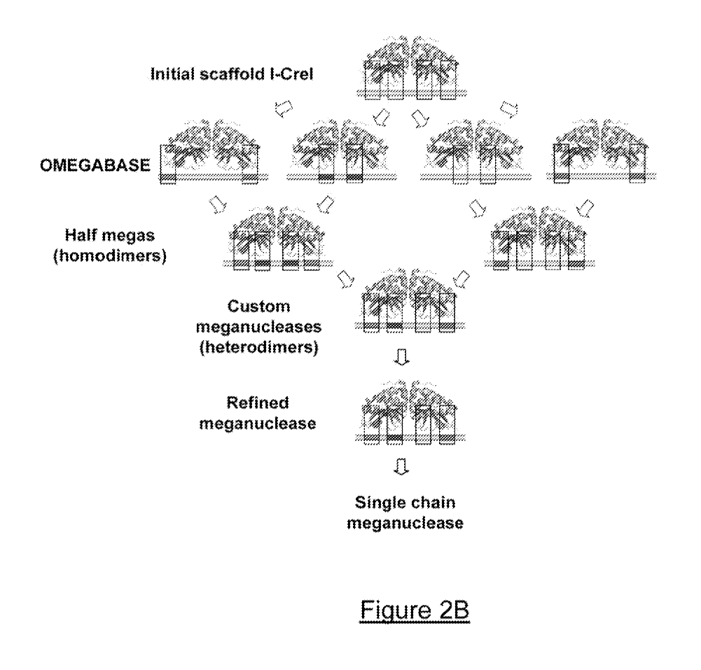

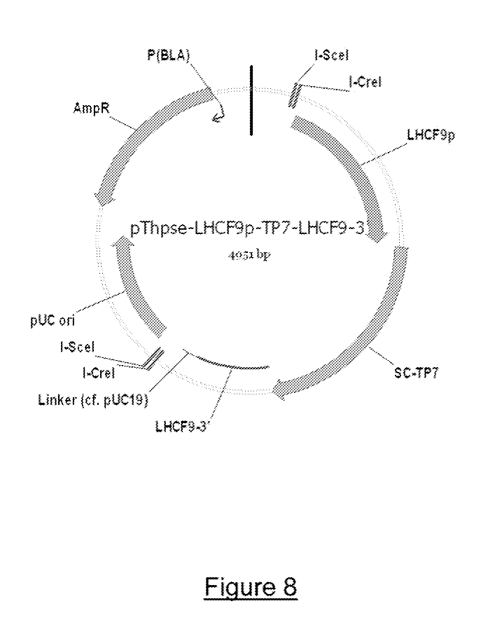

Meganuclease TP7 (TP7) targeting TP07.1 target (SEQ ID NO: 42 in FIG. 6) was obtained according to previously published methods (Grizot et al. 2009) and as described in the legend of FIG. 2. The TP7 single chain ORF (SEQ ID NO: 69) was excised by digestion from the plasmid pCLS7126 (SEQ ID NO: 70) then subcloned by ligation into a diatom specific expression plasmid. The latter contains regulatory regions (a LHCF9p promoter and a LHCF9-3′ terminator) previously cloned into a vector containing only ampicillin resistance cassette and bacterial replication origin. The theoretical map of the resulting plasmid is depicted in FIG. 8 while its complete sequence is listed in SEQ ID NO: 71.

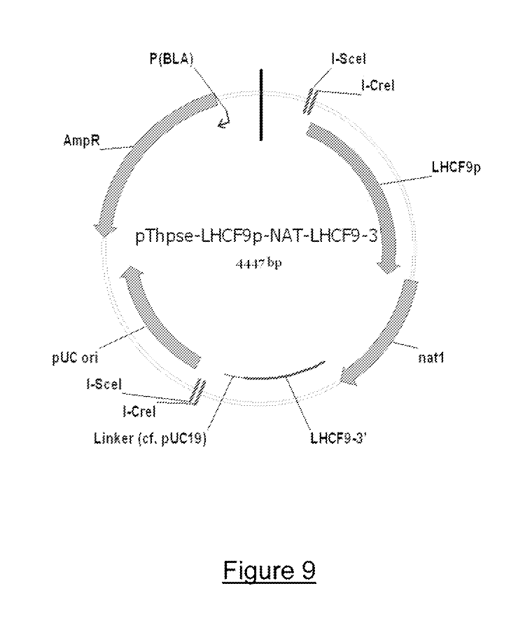

nat1 gene was subcloned into the same diatom specific expression vector between the promoter and the terminator regions. The theoretical map of the resulting plasmid is depicted in FIG. 9 while its complete sequence is listed in SEQ ID NO: 72.

2) Diatom Culture CCMP1335 Transformation

Diatom culture CCMP1335, species Thalassiosira pseudonana was genetically transformed by Cytopulse electroporation technology (Cellectis SA): 10⁷ cells were collected from an exponentially growing culture (concentration not exceeding 10⁶ cells·ml⁻¹) by centrifugation at 2500 rpm for 15 minutes. The supernatant was discarded and the pellet resuspended in 200 μl electroporation buffer to which 3 μg of TP7 expression plasmid (FIG. 8, SEQ ID NO: 71) and 3 μg of nat plasmid (FIG. 9, SEQ ID NO: 72) were previously added. The mix was then transferred to prechilled BioRad electroporation cuvettes (0.4 cm gap). Electroporation was performed in a CytoLVT-S (Cellectis Inc.) using the following program:

4×(1200 V 0.2 ms) 50 ms interval 8×(800 V 0.8 ms)

Group 1 pulses are separated by a 0.2 ms gap. Group 2 pulses are separated by a 2 ms gap.

After electroporation, cuvettes were put back on ice until next step. Electroporated cells were diluted into 2 mL complete growth medium (40 g Sigma Sea Salts in 980 mL sterile MilliQ water+20 ml Sigma F2 enrichment solution), were transferred into a plate well or a 25 cm² flask and were incubated overnight in a growth chamber.

About 5×10⁶ cells are plated onto agar plates filled with 25 mL of solid medium (20 g Sigma Sea Salts in 980 mL sterile MilliQ water+20 ml Sigma F2 enrichment solution+10 g Pure Agar+100 mg nourseothricin).

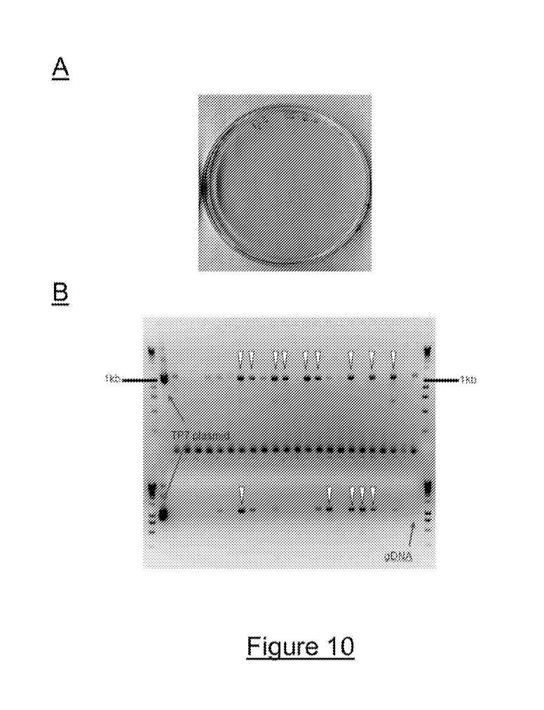

About 400 nourseothricin resistant colonies were recorded upon selection (FIG. 10A). 44 of them were PCR screened for the presence of TP7 meganuclease into the diatom genome (FIG. 10B). Fourteen strains (arrowheads) were transferred to liquid in order to increase algal biomass and isolate, DNA, RNA, and proteins for deep sequencing experiments, meganuclease expression, meganuclease protein accumulation into the cells.

3) DNA, RNA and Protein Isolation

DNA was isolated 41 days after transformation using a modification of the CTAB method (Amato et al., 2007); RNA was extracted 51 days after transformation by Trizol (Invitrogen) and purified by PureLink RNA minikit (Invitrogen), then reverse transcribed by SuperScript III kit (Invitrogen) to obtain cDNAs; proteins were extracted 55 days after transformation with the following protocol:
Collect cells by centrifugation (3000 rpm 15 minutes)
Discard the supernatants and resuspend pellets into lysis buffer (Tris HCl 50 mM, SDS 2%. pH 6.8)
Vortex to destroy cell membranes and leave at room temperature for 30 minutes
Centrifuge, collect the supernatant and quantify proteins by BCA protocol (Pierce).

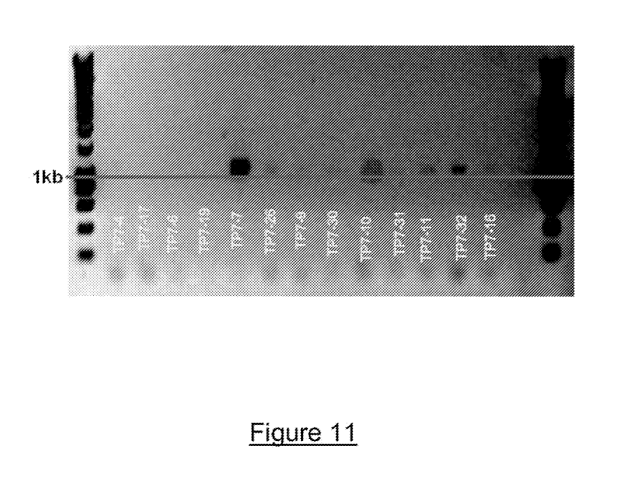

1.6 μg total RNA was reverse transcribed as described before and 1 μl of the cDNAs was amplified using meganuclease-specific primers in order to verify TP7 ORF transcription (FIG. 11). One out of the 13 cultures showed a strong band at around 1 kb (the expected size for TP7 single chain meganuclease). For the other strains, much weaker bands were recorded, but still a relative level of TP7 mRNA was detected.

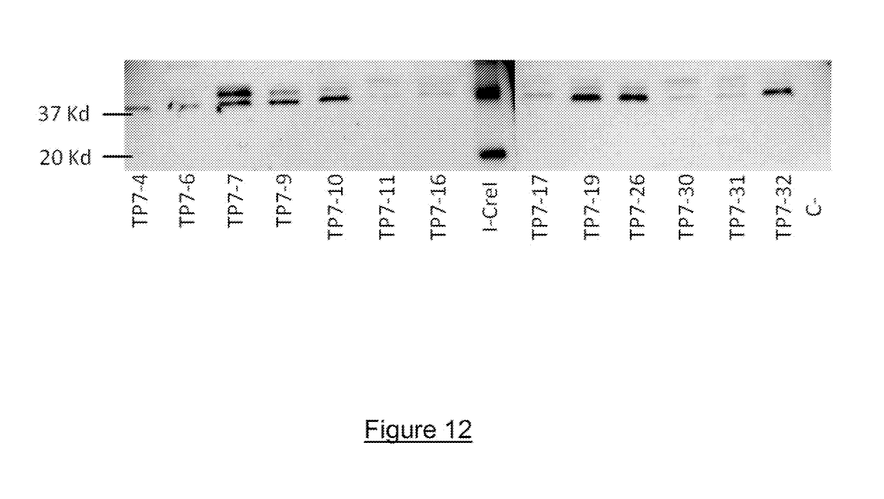

30 μg total protein extracts were loaded on polyacrylamide gel for electrophoretic separation and subsequent western blot analyses (FIG. 12). After electrophoresis, proteins were transferred to nitrocellulose membranes and hybridized with a rabbit polyclonal anti-I-CreI N75 (1:20000) that recognizes all engineered meganucleases (Cellectis SA). Revelation was made using a goat anti-rabbit IgG horseradish peroxidase conjugated secondary antibody (1:5000). Incubation with chemiluminescent Luminol Reagent produces light that is detected on a photographic film.

4) Deep Sequencing Analysis

100 ng genomic DNA of each culture were amplified by PCR using primers listed in Table 1 below. The primer pairs were designed to amplify a region surrounding the TP7 target. 370 bp amplicons were produced containing specific sequences to be bound onto magnetic beads for deep sequencing and specific recognition regions to univocally label each amplicon. Non homologous end-joining (NHEJ) events produced by the meganuclease TP7 were estimated by deep sequencing. TOT sequences were automatically analysed

TABLE 1

| | | SEQ ID |
|---|---|---|
| name | sequence 5′-3′ | NO: |
| MID-129-TP7-4Fw | CCATCTCATCCCTGCGTGTCTCCGACTCAGCAGACGTCTGGACGCAGCATTTAGCCATGAAGGT | 74 |
| MID-130-TP7-6Fw | CCATCTCATCCCTGCGTGTCTCCGACTCAGCAGTACTGCGGACGCAGCATTTAGCCATGAAGGT | 75 |
| MID-131-TP7-7Fw | CCATCTCATCCCTGCGTGTCTCCGACTCAGCGACAGCGAGGACGCAGCATTTAGCCATGAAGGT | 76 |
| MID-132-TP7-9Fw | CCATCTCATCCCTGCGTGTCTCCGACTCAGCGATCTGTCGGACGCAGCATTTAGCCATGAAGGT | 77 |
| MID-133-TP7-10Fw | CCATCTCATCCCTGCGTGTCTCCGACTCAGCGCGTGCTAGGACGCAGCATTTAGCCATGAAGGT | 78 |
| MID-134-TP7-11Fw | CCATCTCATCCCTGCGTGTCTCCGACTCAGCGCTCGAGTGGACGCAGCATTTAGCCATGAAGGT | 79 |
| MID-135-TP7-16Fw | CCATCTCATCCCTGCGTGTCTCCGACTCAGCGTGATGACGGACGCAGCATTTAGCCATGAAGGT | 80 |
| MID-136-TP7-17Fw | CCATCTCATCCCTGCGTGTCTCCGACTCAGCTATGTACAGGACGCAGCATTTAGCCATGAAGGT | 81 |
| MID-137-TP7-19Fw | CCATCTCATCCCTGCGTGTCTCCGACTCAGCTCGATATAGGACGCAGCATTTAGCCATGAAGGT | 82 |
| MID-138-TP7-26Fw | CCATCTCATCCCTGCGTGTCTCCGACTCAGCTCGCACGCGGACGCAGCATTTAGCCATGAAGGT | 83 |
| MID-139-TP7-30Fw | CCATCTCATCCCTGCGTGTCTCCGACTCAGCTGCGTCACGGACGCAGCATTTAGCCATGAAGGT | 84 |
| MID-140-TP7-31Fw | CCATCTCATCCCTGCGTGTCTCCGACTCAGCTGTGCGTCGGACGCAGCATTTAGCCATGAAGGT | 85 |
| MID-141-TP7-32Fw | CCATCTCATCCCTGCGTGTCTCCGACTCAGTAGCATACTGGACGCAGCATTTAGCCATGAAGGT | 86 |
| MID-142-TP7-43Fw | CCATCTCATCCCTGCGTGTCTCCGACTCAGTATACATGTGGACGCAGCATTTAGCCATGAAGGT | 87 |
| MID-143-TP7-wtFw | CCATCTCATCCCTGCGTGTCTCCGACTCAGTATCACTCAGGACGCAGCATTTAGCCATGAAGGT | 88 |
| DeepTP7Rv | CCTATCCCCTGTGTGCCTTGGCAGTCTCAGAGCTCACGCGGGCTCGTCAT | 89 |

14 forward primers and one reverse primer for deep sequencing of a 370 bp amplicon spanning over the TP7 target. Each forward primer contains a MID (Multipled Identifier) region required to univocally recognize the amplicon (marked boldface in table), an adapter to bind the amplicons to magnetic beads (marked plainface in table) and the region-specific sequence (marked italics in table) to prime DNA polymerase replication.

5) Knock-Out by Targeted Knock-in

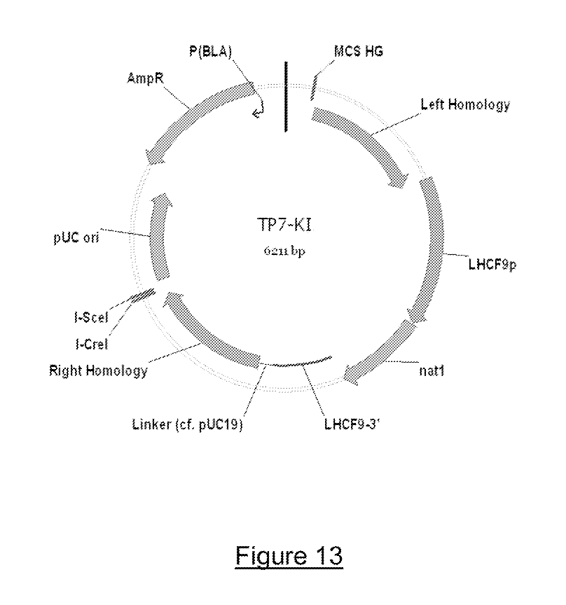

Using the meganuclease TP7 that hits on chromosome 3 into the gene encoding for the protein ID: 261853 a knock-in is produced by co-transfecting the TP7 expression plasmid (FIG. 8, SEQ ID NO: 71) and the knock-in matrix (FIG. 13, SEQ ID NO: 73). The latter plasmid is constructed by cloning two 920 bp-regions flanking the TP7 target (SEQ ID NO: 73), about 400 bp up- and downstream this locus. The left homology (located upstream the TP7 target) is amplified by PCR from the genome of the diatom T. pseudonana using the forward primer NotI-TP7LH-Fw (SEQ ID NO: 90, Table 2 below) and the reverse primer PstI-TP7LH-Rv (SEQ ID NO: 91, Table 2 below). The former primer introduced a NotI restriction site in 5′ while the latter a PstI restriction site in 3′. This region is cloned by digestion-ligation into the nat plasmid (FIG. 9, SEQ ID NO: 72) upstream the nat1 gene expression cassette giving rise to the TP7-LH plasmid. Following the same strategy, the right homology (located downstream the TP7 target) is amplified by PCR from the T. pseudonana genome using the primers EcoRI-TP7RH-Fw (SEQ ID NO: 92) and AflII-TP7RH-Rv (SEQ ID NO: 93). EcoRI and AflII are the 5′ and 3′ restriction sites carried by the forward and reverse primer respectively. The TP7-LH plasmid is digested by MfeI and AflII enzymes, the PCR product by EcoRI and AflII (note that EcoRI and MfeI are compatible enzymes, both leaving an AATT sticky end) then ligated to produce the TP7-KI matrix (FIG. 13, SEQ ID NO: 73).

TABLE 2

| | | organism or | sequence 5′-3′ |
|---|---|---|---|
| name | notes | plasmid | (restriction enzyme site in bold) |
| NotI-TP7LH-Fw | carries a NotI site in 5′ | Thalassiosira | atatgcggccgccaagcttcatttgttggccg |
| | | pseudonana | (SEQ ID NO: 90) |
| PstI-TP7LH-Rv | Cerries a PstI site in 5′ | Thalassiosira | ttaactgcagtgacgagcccccgtgagctg |
| | | pseudonana | (SEQ ID NO: 91) |
| EcoRI-TP7RH-Fw | Carries a EcoRI site in 5′ | Thalassiosira | atatgaattctcgcttggagctatcattac |
| | to be cloned in the MfeI- | pseudonana | (SEQ ID NO: 92) |
| | AflII fragment of the nat | | |
| | plasmid | | |
| AflII-TP7RH-Rv | carries a AflII site in 5′ | Thalassiosira | ttaacttaagatgagaacaggtgaattggcgg |
| | | pseudonana | (SEQ ID NO: 93) |

list of primers used for cloning of left and right homologies in the knock-in matrix.

10⁷ cells are co-electroporated following the protocol described above in paragraph 2) with the two plasmids nat (SEQ ID NO: 72) and TP7-KI (SEQ ID NO: 73). The day after electroporation, cells are plated onto selective plates (100 μg nourseothricin). Nourseothricin-positive colonies are screened by PCR in order to check whether the nat1 gene is randomly integrated or is integrated between the left and right homology into the gene.

### LIST OF REFERENCE CITED IN THE DESCRIPTION

Norton, T. A., Melkonian, M., & Andersen, R. A. (1996) Phycologia 35, 308-326
Steinbrenner, J. & Sandmann, G. (2006) Appl. Environ. Microbiol. 72, 7477-7484
Mogedas, B., Casal, C., Forjan, E., & Vilchez, C. (2009) J Biosci Bioeng 108, 47-51.
Palmer, J. D., Soltis, D. E., & Chase, M. W. (2004) Am. J. Bot. 91, 1437-1445
Tyler, B. M., Tripathy, S., Zhang, X., Dehal, P., Jiang, R. H. Y., Aerts, A., Arredondo, F. D., Baxter, L., Bensasson, D., Beynon, J. L. et al. (2006) Science 313, 1261-1266
Armbrust, E. V., Berges, J. A., Bowler, C., Green, B. R., Martinez, D., Putnam, N. H., Zhou, S., Allen, A. E., Apt, K. E., Bechner, M. et al. (2004) Science 306, 79-86.
Bowler, C., Allen, A. E., Badger, J. H., Grimwood, J., Jabbari, K., Kuo, A., Maheswari, U., Martens, C., Maumus, F., Otillar, R. P. et al. (2008) Nature. 456, 239-244.
Bowler, C., Allen, A. E., Badger, J. H., Grimwood, J., Jabbari, K., Kuo, A., Maheswari, U., Martens, C., Maumus, F., Otillar, R. P. et al. (2008) Nature. 456, 239-244.
Lusic et al Advanced Functional Materials 2006
Rouet et al. PNAS 1994 91:6064-6068
Rouet et al. Mol Cell Biol. 1994 14 :8096-8106
Choulika et al. Mol Cell Biol. 1995 15 :1968-1973
Puchta et al. PNAS 1996 93 :5055-5060
Paques et al. Curr Gen Ther. 2007 7:49-66
Arnould et al. J Mol Biol. 2007 371:49-65
Grizot et al. NAR 2009 37:5405
Galetto et al. Expert Opin Biol Ther. 2009 9:1289-303
Norton, T. A., Melkonian, M., & Andersen, R. A. (1996) Phycologia 35, 308-326
Steinbrenner, J. & Sandmann, G. (2006) Appl. Environ. Microbiol. 72, 7477-7484
Mogedas, B., Casal, C., Forjan, E., & Vilchez, C. (2009) J Biosci Bioeng 108, 47-51.
Palmer, J. D., Soltis, D. E., & Chase, M. W. (2004) Am. J. Bot. 91, 1437-1445
Tyler, B. M., Tripathy, S., Zhang, X., Dehal, P., Jiang, R. H. Y., Aerts, A., Arredondo, F. D., Baxter, L., Bensasson, D., Beynon, J. L. et al. (2006) Science 313, 1261-1266
Armbrust, E. V., Berges, J. A., Bowler, C., Green, B. R., Martinez, D., Putnam, N. H., Zhou, S., Allen, A. E., Apt, K. E., Bechner, M. et al. (2004) Science 306, 79-86.
Bowler, C., Allen, A. E., Badger, J. H., Grimwood, J., Jabbari, K., Kuo, A., Maheswari, U., Martens, C., Maumus, F., Otillar, R. P. et al. (2008) Nature. 456, 239-244.
Bowler, C., Allen, A. E., Badger, J. H., Grimwood, J., Jabbari, K., Kuo, A., Maheswari, U., Martens, C., Maumus, F., Otillar, R. P. et al. (2008) Nature. 456, 239-244.
Lusic et al Advanced Functional Materials 2006
Rouet et al. PNAS 1994 91:6064-6068
Rouet et al. Mol Cell Biol. 1994 14 :8096-8106
Choulika et al. Mol Cell Biol. 1995 15 :1968-1973
Puchta et al. PNAS 1996 93 :5055-5060
Paques et al. Curr Gen Ther. 2007 7:49-66
Arnould et al. J Mol Biol. 2007 371:49-65
Grizot et al. NAR 2009 37:5405
Galetto et al. Expert Opin Biol Ther. 2009 9:1289-303
Paques et al. Curr Gen Ther. 2007 7:49-66
Arimondo et al. Mol Cell Biol. 2006 26:324-333; Simon et al. NAR 2008 36:3531-3538
Porteus et al. Nat Biotechnol. 2005 23:967-973
Cannata et al. PNAS 2008 105:9576-9581
Lee et al., 2002, EMBO J. 21: 4663-4670
Lee et al, 2003, Nature 425:415-419
Yi et al, 2003, Genes and Development 17:3011-3016
Hutvagner et al, 2001, Science 293:834-838
Bartel et al, 2004, Cell 116: 281-297
Bouvier-Nave et al. 2000, European J. of Biochem., Vol. 267, pp. 85-96.
Cheng et al. 2006, Gene. Vol. 371, pp. 112-120.
Desbois, et al. 2009, Mar Biotechnol. (NY), Vol. 11, pp. 45-52.
Dunahay et al. 1996, Appl. Biochem. Biotechnol., Vol. 57, pp. 223-231.
Frommolt et al. 2008, Mol. Biol. Evol., Vol. 25, pp. 2653-2667.
Gordon et al. 2008, Trends in Biotechnology, Vol. 27, pp. 116-127.
Hentzer and Givskov. 2003, J. Clin Invest., Vol. 112, pp. 1300-1307.
Jako et al. 2001, Plant Physiology, Vol. 126, pp. 861-874.
Junchao et al. 2008, J. of Phycology, Vol. 44, pp. 684-690.
Kuehl et al. 2009, Antimicrobial Agents and Chemotherapy, Vol. 53, pp. 4159-4166.
Li et al. 2010, Metabolic Engineering, Vol. 12, pp. 387-391.
Li et al. 2010, Biotechnology and Bioengineering. Pub online May 20 (Epub ahead of print).
Marillia et al. 2003, J. of Exp. Botany, Vol. 54, pp. 259-270
Roesler et al. 1997, Plant Physiology, Vol. 113, pp. 75-81.
Teplitski et al. 2004, Plant Physiology, Vol. 134, pp. 137-146.
Thamatrakoln and Hildebrand. 2008, Plant Physiology, Vol. 146, pp. 1397-1407.
Vigeolas et al. 2007, Plant Biotech. J., Vol. 5, pp. 431-441.
Weselake et al. 2008, J. of Exp. Botany, Vol. 59, pp. 3543-3549.
Williams P. 2007, Microbiology, Vol. 153, pp. 3923-3938.
Zabawinski et al. 2001, J. of Bacteriology, Vol. 183, pp. 1069-1077.
Zheng et al. 2008, Nature Genetics, Vol. 40, pp. 367-372.
Zou et al. 1997, Plant Cell, Vol. 9, pp. 909-923.
Amato et al. 2007, Protist 158:193-207.

1-15. (canceled)

16. A method for targeted genomic engineering in an algal cell comprising introducing an endonuclease into the algal cell to induce a double-stranded cleavage at a site of interest in the genome of the algal cell.

17. The method of claim 16, comprising:
providing an endonuclease capable of inducing a double-stranded cleavage at a site of interest in the genome of an algal cell;
introducing the endonuclease into an algal cell; and
isolating an algal cell having a modified targeted genomic site of interest.

18. The method of claim 16, wherein the endonuclease is introduced into the algal cell by electroporation or bombardment.

19. The method of claim 17, wherein the endonuclease is introduced into the algal cell by electroporation or bombardment.

20. The method of claim 16, wherein a targeted knock-out in algae is induced by the endonuclease at the site of interest in the genome.

21. The method of claim 16, wherein at least one transgene is inserted at the targeted genomic site of interest by introducing a template that is flanked by sequences sharing homology with the region surrounding the genomic DNA cleavage site of interest.

22. The method of claim 21, wherein the template comprises at least one transgene encoding a gene selected from the group consisting of quorum sensing, secretion of hydrocarbons, fatty acid composition, lipids accumulation, enhanced photosynthesis, pigments production, mercury volatilization, frustule composition or organization, and mitigation genes.

23. The method of claim 21, wherein the template comprises a nucleic acid encoding a selectable marker.

24. The method of claim 23, wherein the selectable marker is N-acetyltransferase 1 (Nat1) conferring the resistance to Nourseothricin.

25. The method of claim 22, wherein the transgene insertion does not modify expression of genes located in the vicinity of the target sequence.

26. The method of claim 21, wherein the template comprises multiple transgenes.

27. The method of claim 16, wherein the endonuclease is a meganuclease.

28. The method of claim 27, wherein the meganuclease is selected from homodimers, heterodimers, obligate heterodimers and single chain variants.

29. The method of claim 27, wherein the meganuclease is an engineered I-CreI.

30. The method of claim 17, wherein the endonuclease is an engineered zinc-finger binding domain fused to a restriction enzyme.

31. The method of claim 17, wherein the algal cell is selected from the group consisting of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium algal cells.

32. A targeted genome engineered algae obtained by the method of claim 16.

33. The targeted genome engineered algae of claim 32, comprising at least one transgene inserted into a targeted genomic site of interest.

34. The targeted genome engineered algae of claim 33, wherein the transgene encodes a gene selected from the group consisting of quorum sensing, secretion of hydrocarbons, fatty acid composition, lipids accumulation, enhanced photosynthesis, pigments production, mercury volatilization, frustule composition or organization, and mitigation genes.

35. An algae comprising a nucleic acid sequence encoding an endonuclease.

36. A method of increasing biofuel production comprising introducing an endonuclease into an algal cell to induce a double-stranded cleavage within a gene regulating the production of fatty acid and triacylglcerols in the genome of the algal cell,
wherein the cleavage results in an increase of fatty acid and triacylglcerols in the algal cell.

37. The method according to claim 36 comprising:
providing an endonuclease capable of inducing a double-stranded cleavage at a site of interest in the genome of an algal cell;
introducing the endonuclease into an algal cell; and
isolating an algal cell having a modified targeted genomic site of interest.

38. The method of claim 37, wherein the endonuclease is introduced into the algal cell by electroporation or bombardment.


SEQUENCE LISTING (93 sequences)

SEQ ID NO: 1 - protein, 163 aa, Chlamydomonas reinhardtii monomer
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - protein, 167 aa, Chlamydomonas reinhardtii monomer
MANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 3 - DNA, 22 bp, Artificial Sequence
  C1221 target
caaaacgtcgtacgacgttttg

SEQ ID NO: 4 - DNA, 24 bp, Artificial Sequence
  STA6.1 target
tgggcacgacttgcattgtgtact

SEQ ID NO: 5 - DNA, 24 bp, Artificial Sequence
  STA6.2 target
atttactgcctcacccagttcaac

SEQ ID NO: 6 - DNA, 24 bp, Artificial Sequence
  STA6.3 target
tcccgctagggcacaggagcgaac

SEQ ID NO: 7 - DNA, 24 bp, Artificial Sequence
  STA6.4 target
accgcacaccgtaccgcgtccaca

SEQ ID NO: 8 - DNA, 24 bp, Artificial Sequence
  STA6.5 target
gtccgccaacaagagctggttcca

SEQ ID NO: 9 - DNA, 24 bp, Artificial Sequence
  STA6.6 target
ttcctcctgcgcaaggcagcgagg

SEQ ID NO: 10 - DNA, 24 bp, Artificial Sequence
  STA6.7 target
tgaggccgccgtaactgggggtgg

SEQ ID NO: 11 - DNA, 24 bp, Artificial Sequence
  STA6.8 target
gtgccactgggcacggtggcctgc

SEQ ID NO: 12 - DNA, 24 bp, Artificial Sequence
  STA6.9 target
acgcgctggtacacggagggctac

SEQ ID NO: 13 - DNA, 24 bp, Artificial Sequence
  STA6.10 target
tggggatatggttccagggctaat

SEQ ID NO: 14 - DNA, 24 bp, Artificial Sequence
  STA6.11 target
ctgggatgggtcaaggtggagggg

SEQ ID NO: 15 - DNA, 24 bp, Artificial Sequence
  STA6.12 target
acgcgccgatctacaccatgtcgc

SEQ ID NO: 16 - DNA, 24 bp, Artificial Sequence
  STA6.13 target
tcgggccgggaagaggcggcgcgg

SEQ ID NO: 17 - DNA, 24 bp, Artificial Sequence
  STA6.14 target
cttggctgcgtttttgggttggaa

SEQ ID NO: 18 - DNA, 24 bp, Artificial Sequence
  STA6.15 target
actctatagagtaggggggattga

SEQ ID NO: 19 - DNA, 24 bp, Artificial Sequence
  STA6.16 target
gtgggatgccgtaggaggggcggg

SEQ ID NO: 20 - DNA, 24 bp, Artificial Sequence
  chr_2 target
tcgtgatgctgtaaaggattttga

SEQ ID NO: 21 - DNA, 24 bp, Artificial Sequence
  chr_2 target
ttttgacgtcgtacggtgtctccg

SEQ ID NO: 22 - DNA, 24 bp, Artificial Sequence
  chr_4 target
gaaggataccgtaagtaggtttgg

SEQ ID NO: 23 - DNA, 24 bp, Artificial Sequence
  chr_4 target
acaagccattgtacgttgttccgt

SEQ ID NO: 24 - DNA, 24 bp, Artificial Sequence
  chr_4 target
attgaatcttttacaagagcaagg

SEQ ID NO: 25 - DNA, 24 bp, Artificial Sequence
  chr_7 target
ttttgctcttgtacggcgtcctgg

SEQ ID NO: 26 - DNA, 24 bp, Artificial Sequence
  chr_8 target
tcaaaactttgtacagaatgttgt

SEQ ID NO: 27 - DNA, 24 bp, Artificial Sequence
  chr_9 target
accagaccccgtaaaagagatgga

SEQ ID NO: 28 - DNA, 24 bp, Artificial Sequence
  chr_9 target
acgaaactacgtactccattttgg

SEQ ID NO: 29 - DNA, 24 bp, Artificial Sequence
  chr_12 target
gaaaactgtcgtacagggtctcga

SEQ ID NO: 30 - DNA, 24 bp, Artificial Sequence
  chr_16 target
gcaaactctgttacatagtacaac

SEQ ID NO: 31 - DNA, 24 bp, Artificial Sequence
  chr_17 target
gcaaactgagttaagggatccgaa

SEQ ID NO: 32 - DNA, 24 bp, Artificial Sequence
  chr_18 target
ccgagaccctgtaaaaggtggaag

SEQ ID NO: 33 - DNA, 24 bp, Artificial Sequence
  chr_18 target
cctggccctggtaaatagtcttgg

SEQ ID NO: 34 - DNA, 24 bp, Artificial Sequence
  chr_18 target
tttgtcttttgtaagacagacaat

SEQ ID NO: 35 - DNA, 24 bp, Artificial Sequence
  chr_19 target
acaaactggcgtaacgcagtttat

SEQ ID NO: 36 - DNA, 24 bp, Artificial Sequence
  chr_20 target
ccaaaccgttttacagtatattca

SEQ ID NO: 37 - DNA, 24 bp, Artificial Sequence
  chr_21 target
acgggactacgtacgacggaatgc

SEQ ID NO: 38 - DNA, 24 bp, Artificial Sequence
  chr_21 target
gcaaaatggtttacgacagtacga

SEQ ID NO: 39 - DNA, 24 bp, Artificial Sequence
  chr_28 target
gttttacgttgtacgacgtctagc

SEQ ID NO: 40 - DNA, 24 bp, Artificial Sequence
  chr_2 target
tccttcttcggtacgtcattttct

SEQ ID NO: 41 - DNA, 24 bp, Artificial Sequence
  chr_3 target
acaagacgtctcacgttgttccgt

SEQ ID NO: 42 - DNA, 24 bp, Artificial Sequence
  chr_3 target
tttggccgaggtacagggtacaaa

SEQ ID NO: 43 - DNA, 24 bp, Artificial Sequence
  chr_3 target
gctacatgtcgtaaccagtacgga

SEQ ID NO: 44 - DNA, 24 bp, Artificial Sequence
  chr_4 target
acaatatggcttacaaaagacagg

SEQ ID NO: 45 - DNA, 24 bp, Artificial Sequence
  chr_5 target
acaagctattttacaagggtcggc

SEQ ID NO: 46 - DNA, 24 bp, Artificial Sequence
  chr_6 target
tccctctcttgtgacaaatgtaaa

SEQ ID NO: 47 - DNA, 24 bp, Artificial Sequence
  chr_6 target
tcctgctcctgtaagaaagattgc

SEQ ID NO: 48 - DNA, 24 bp, Artificial Sequence
  chr_6 target
ataggatccatcacgacgtttcac

SEQ ID NO: 49 - DNA, 24 bp, Artificial Sequence
  chr_8 target
actggccgatgtaagacgtccaac

SEQ ID NO: 50 - DNA, 24 bp, Artificial Sequence
  chr_8 target
gtgaaactatgtaaaaaggcaatt

SEQ ID NO: 51 - DNA, 24 bp, Artificial Sequence
  chr_13 target
atgaaacgacgtacgaagtattgg

SEQ ID NO: 52 - DNA, 24 bp, Artificial Sequence
  chr_13 target
tcaatctcaggtgaaacagagcgt

SEQ ID NO: 53 - DNA, 24 bp, Artificial Sequence
  chr_14 target
tctggctctggtacgttatcttgt

SEQ ID NO: 54 - DNA, 24 bp, Artificial Sequence
  chr_15 target
atgacatccgttaccgaagaagat

SEQ ID NO: 55 - DNA, 24 bp, Artificial Sequence
  chr_15 target
attagacgaggtgagacgtcttgt

SEQ ID NO: 56 - DNA, 24 bp, Artificial Sequence
  chr_16a target
acaggacgatgtacgacagcgcag

SEQ ID NO: 57 - DNA, 24 bp, Artificial Sequence
  chr_17 target
tcaaccttctttacaagatctgac

SEQ ID NO: 58 - DNA, 24 bp, Artificial Sequence
  chr_23 target
gtgatacgttgtgagaaagattaa

SEQ ID NO: 59 - DNA, 24 bp, Artificial Sequence
  scaffold_1 target
attttatgttgtgaagaaggttgg

SEQ ID NO: 60 - DNA, 24 bp, Artificial Sequence
  scaffold_1 target
accttctccattaccccatccccc

SEQ ID NO: 61 - DNA, 24 bp, Artificial Sequence
  scaffold_12 target
gcgacctctggtgaggggttttgc

SEQ ID NO: 62 - DNA, 24 bp, Artificial Sequence
  scaffold_19 target
agggcctgtggtacgacgtctggg

SEQ ID NO: 63 - DNA, 24 bp, Artificial Sequence
  scaffold_20 target
acaaaacgtcgtacaggatgcaag

SEQ ID NO: 64 - DNA, 24 bp, Artificial Sequence
  scaffold_20 target
cctgcctgttttaccccgttttgg

SEQ ID NO: 65 - DNA, 24 bp, Artificial Sequence
  scaffold_24 target
actgcctgttttacaacgtgcagc

SEQ ID NO: 66 - DNA, 24 bp, Artificial Sequence
  scaffold_32 target
acctgcccctgtacccaggcttgc

SEQ ID NO: 67 - DNA, 24 bp, Artificial Sequence
  scaffold_33 target
attgaactttgtaagtagttttgc

SEQ ID NO: 68 - DNA, 24 bp, Artificial Sequence
  scaffold_40 target
gcttcatcatgtacaacggatcac

SEQ ID NO: 69 - protein, 349 aa, Artificial Sequence
  TP7 single chain ORF
MANTKYNEEFLLYLAGFVDGDGSIIAQIKPNQSTKFKHALKLTFNVTQKTQRRWFLDKLVDEIGVGYVYDSGSVSYYNLSEIKPLHNFLTQLQPFLELKQKQANLVLKIIEQLPSAKESPDKFLEVCTWVDQVAALNDSKTRKTTSETVRAVLDSLSEKKKSSPAAGDSSVSNSEHIAPLSLPSSPPSVGSNKKFLLYLAGFVDSDGSIIAQIQPNQSSKFKHRLKLTFKVTQKTQRRWLLDKLVDRIGVGYVEDSGSVSNYRLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 70 - DNA, 3829 bp, Artificial Sequence
  pCLS7126
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatattgggagctcgtctagaggatcgctcgagttactaaggagaggactttttcttctcagagaggctatccagaactgccctcacagtctcagaggtggtttttctggtcttggagtcattcaaggcagcaacctgatccacccaagtacacacttcaagaaacttgtcaggggactccttggcagatggcagttgctcgatgattttcaaaaccagatttgcttgcttctgtttgagcttcaagaagggttgcagttgggtgagaaagttatgaagaggcttaatttcagacagacggtagtttgacacagagccagagtcttcgacatagcccacaccaatacgatcaaccaatttgtccaacagccaccttctttgtgtcttctgagtgactttaaaggtcaatttgagacggtgtttgaacttagaagattgatttggctgtatctgagcaatgatggagccatcagaatccacaaatccagcaagatacagcaggaattttttgttagaaccaacagatggaggagaggaaggcagagacagaggagcaatgtgctcggaattagaaacagaggaatcaccggccgccggggaggatttcttcttctcgctcaggctgtccagcacagcacgaacggtttcagaagtggttttacgcgtcttagaatcgttcagagctgcaacctgatccacccaggtacaaacttccaggaatttgtccggggattcttttgcagacggcagctgttcgataattttcagaaccaggtttgcctgtttctgtttcagttccagaaacggctgcagttgagtcaggaagttgtgcagcggcttgatttcgcttaagttgtagtaggaaacgctaccagaatcgtatacgtaaccaacgccaatttcatccactagtttgtccagaaaccaacggcgctgggtcttttgagtcacgttaaaggtcaattttagagcatgtttaaacttggtagactggtttggtttaatctgagcgatgatgctaccgtcaccgtccacaaagccggccaggtacagcaggaactcttcgttatatttggtattggccatggtggcaaggccgctgtgtaggcgcgccattgggtcatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 71 - DNA, 4951 bp, Artificial Sequence
  pThpse-LHCF9p-TP7-LHCF9-3′ expression plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaattaattaagtaggcgcgcctacacagcggccttgccaccatggccaataccaaatataacgaagagttcctgctgtacctggccggctttgtggacggtgacggtagcatcatcgctcagattaaaccaaaccagtctaccaagtttaaacatgctctaaaattgacctttaacgtgactcaaaagacccagcgccgttggtttctggacaaactagtggatgaaattggcgttggttacgtatacgattctggtagcgtttcctactacaacttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtgattcctctgtttctaattccgagcacattgctcctctgtctctgccttcctctcctccatctgttggttctaacaaaaaattcctgctgtatcttgctggatttgtggattctgatggctccatcattgctcagatacagccaaatcaatcttctaagttcaaacaccgtctcaaattgacctttaaagtcactcagaagacacaaagaaggtggctgttggacaaattggttgatcgtattggtgtgggctatgtcgaagactctggctctgtgtcaaactaccgtctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttagtaactcgagcgatcctctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctcccgtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 72 - DNA, 4447 bp, Artificial Sequence
  pThpse-LHCF9p-NAT-LHCF9-3′ expression plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgcgctagggataacagggtaatattcaaaacgtcgtacgacgttttgacctgcaggcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaattaattaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctgaggcgcgccttaacatgtttgctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctcccgtttaaactcactgactcgctgcgctcggtcgttcggctgcggcgagcggtatcagctcactcaaaggcggtaatacggttatccacagaatcaggggataacgcaggaaagacaattgcttataacacgcgtactagtgctcgcgacgagatcttacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 73 - DNA, 6211 bp, Artificial Sequence
  TP7-KI matrix
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgatgcatccgttaacaccggtaagcggccgccaagcttcatttgttggccgacaagtgcttgcctcccattttggaagtcatgaatggattggtgggagggaatgacgggaatgagaacgaggactgtgcattacgagctagtttggaggcattggaaaagaccgcagaggaactgtccaagggggtgagccttgctgaagcagtgattgactttgattctgctccccgcgactttatcgtgaatccaaaccttgacagcaaccttggcaacgtcaaggctgaattggatggtattcagcaggagctggaggagatccacgcagaaatgaatgctgcatggtatgaagtgagcaaggagggaggcatgcaacaagttcggctggaggatgttgactcaaatagcaacacttcttgtgtgtggcagtttcgtctacccaaaaccaatgacgcgaagatgttgacggattcttttgattccgttaagatccatcgtattttgaagaacggaggtacgtctcgctttgagtcagcattgttttgggagagcgtccagatattgatgtgtgtctaactactaactactctttttcgctgcttagtgtatttctccacgaaggagttggaacagctcggcacaaagaagaaggatttgatgatggagtacgaggagaagcaacgtgatattgtatgcaaggccatggttgtggctgcaagttacgtgccagtgttggaacgagcatcgatgactttatctgagttggatgtattggcgagctttgcttatgtggctgcatacagtagcaacggatactgtcgtcctgagatgacggacggagaagaggacggattgggaattgaggttagttattcgagcaccgaacgttgcgattcttcgctttggttctctaaacacaacatatcttttctcgtcaatacatttcagctcacgggggctcgtcactgcaggcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccaataccaaattaattaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctgaggcgcgccttaacatgtttgctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattacgttaagtctccttcgtctttggtgtaggcatgttggacacaacgaggtaaaacacaacacaaacaatgtgtccagcaaagtagtagctgctccagttctcccgtttaaactcactgactcgctgcgctcggtcgttcggctgcggcgagcggtatcagctcactcaaaggcggtaatacggttatccacagaatcaggggataacgcaggaaagacaattctcgcttggagctatcattaccttggcgcagattggaagttttgtaccctgtacctcggccaaaatcaacattgttgatcatatcttggccagagttggagctggtgacgcacaggatcgtggtatatctaccttcatggctgaaatgctggaggcttcttccattcttcgcacctcgaccaaacgcagtctcatcatcattgacgagctcggaagaggaacgagcacatttgatggatttggtttggcaaaagcgatatcagaacatgtcgttcagaaaattggttgcatgactgtgtttgcaactcacttccatgaactgacggcgttggaagagcaagaggcctcggtaaccaattgccacgtgtctgcccacagcgacaaacagaacggacttacgtttctctacgaagtacgaccagggccttgcttggaaagttttggtattcaagtggccgaaatggcaaacatgccgtcaaatatcatcaccgatgccaaacgcaaagcaaaacagttggagaactttgactatcgcaagaaagctaaagttacggagaaagactgcatcgctgacaacgaggatgatcatgagacgaaagcagctgcaatggaatttcttcacaagtttaggaagcttccagtgaatgaaatgtcggaagaagagttgaaggagatagcgcttcctttgctaaggcagtacggatttgaagcgttggggtgaagtgatcttgttcagtggatctattcttcatactatctcttcttttgtagtgtaatccatgtaagacttgcttttatgatactgacactatctttcagaactttccgtttgttttgcactgtctctttacgttaggttgccaagcaaaaaaccgttacctgtccgagccagcccccgccaattcacctgttctcataaacttaagcagtcgacaacctaggattagcgctccggtacctcaaaacgtcgtacgacgttttgagctagggataacagggtaatatggatccaagatatcaagaattcccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 74 - DNA, 64 bp, Artificial Sequence
  MID-129-TP7-4Fw primer
ccatctcatccctgcgtgtctccgactcagcagacgtctggacgcagcatttagccatgaaggt

SEQ ID NO: 75 - DNA, 64 bp, Artificial Sequence
  MID-130-TP7-6Fw primer
ccatctcatccctgcgtgtctccgactcagcagtactgcggacgcagcatttagccatgaaggt

SEQ ID NO: 76 - DNA, 64 bp, Artificial Sequence
  MID-131-TP7-7Fw primer
ccatctcatccctgcgtgtctccgactcagcgacagcgaggacgcagcatttagccatgaaggt

SEQ ID NO: 77 - DNA, 64 bp, Artificial Sequence
  MID-132-TP7-9Fw primer
ccatctcatccctgcgtgtctccgactcagcgatctgtcggacgcagcatttagccatgaaggt

SEQ ID NO: 78 - DNA, 64 bp, Artificial Sequence
  MID-133-TP7-10Fw primer
ccatctcatccctgcgtgtctccgactcagcgcgtgctaggacgcagcatttagccatgaaggt

SEQ ID NO: 79 - DNA, 64 bp, Artificial Sequence
  MID-134-TP7-11Fw primer
ccatctcatccctgcgtgtctccgactcagcgctcgagtggacgcagcatttagccatgaaggt

SEQ ID NO: 80 - DNA, 64 bp, Artificial Sequence
  MID-135-TP7-16Fw primer
ccatctcatccctgcgtgtctccgactcagcgtgatgacggacgcagcatttagccatgaaggt

SEQ ID NO: 81 - DNA, 64 bp, Artificial Sequence
  MID-136-TP7-17Fw primer
ccatctcatccctgcgtgtctccgactcagctatgtacaggacgcagcatttagccatgaaggt

SEQ ID NO: 82 - DNA, 64 bp, Artificial Sequence
  MID-137-TP7-19Fw primer
ccatctcatccctgcgtgtctccgactcagctcgatataggacgcagcatttagccatgaaggt

SEQ ID NO: 83 - DNA, 64 bp, Artificial Sequence
  MID-138-TP7-26Fw primer
ccatctcatccctgcgtgtctccgactcagctcgcacgcggacgcagcatttagccatgaaggt

SEQ ID NO: 84 - DNA, 64 bp, Artificial Sequence
  MID-139-TP7-30Fw primer
ccatctcatccctgcgtgtctccgactcagctgcgtcacggacgcagcatttagccatgaaggt

SEQ ID NO: 85 - DNA, 64 bp, Artificial Sequence
  MID-140-TP7-31Fw primer
ccatctcatccctgcgtgtctccgactcagctgtgcgtcggacgcagcatttagccatgaaggt

SEQ ID NO: 86 - DNA, 64 bp, Artificial Sequence
  MID-141-TP7-32Fw primer
ccatctcatccctgcgtgtctccgactcagtagcatactggacgcagcatttagccatgaaggt

SEQ ID NO: 87 - DNA, 64 bp, Artificial Sequence
  MID-142-TP7-43Fw primer
ccatctcatccctgcgtgtctccgactcagtatacatgtggacgcagcatttagccatgaaggt

SEQ ID NO: 88 - DNA, 64 bp, Artificial Sequence
  MID-143-TP7-wtFw primer
ccatctcatccctgcgtgtctccgactcagtatcactcaggacgcagcatttagccatgaaggt

SEQ ID NO: 89 - DNA, 50 bp, Artificial Sequence
  DeepTP7Rv primer
cctatcccctgtgtgccttggcagtctcagagctcacgcgggctcgtcat

SEQ ID NO: 90 - DNA, 32 bp, Artificial Sequence
  NotI-TP7LH-Fw primer
atatgcggccgccaagcttcatttgttggccg

SEQ ID NO: 91 - DNA, 30 bp, Artificial Sequence
  PstI-TP7LH-Rv primer
ttaactgcagtgacgagcccccgtgagctg

SEQ ID NO: 92 - DNA, 30 bp, Artificial Sequence
  EcoRI-TP7RH-Fw primer
atatgaattctcgcttggagctatcattac

SEQ ID NO: 93 - DNA, 32 bp, Artificial Sequence
  AflII-TP7RH-Rv primer
ttaacttaagatgagaacaggtgaattggcgg